(19) United States
(12) Patent Application Publication
(10) Pub. No.: US 2023/0257350 A1
(43) Pub. Date: Aug. 17, 2023

(54) COMPOUNDS AND METHODS OF PROMOTING OLIGODENDROCYTE PRECURSOR DIFFERENTIATION

(71) Applicant: The Cleveland Clinic Foundation, Cleveland, OH (US)

(72) Inventors: Bruce Trapp, Cleveland, OH (US); Satish Medicetty, Cleveland, OH (US); Chunyang Brian Bai, Cleveland, OH (US)

(21) Appl. No.: 18/003,807

(22) PCT Filed: Jul. 2, 2021
(86) PCT No.: PCT/US2021/040294

§ 371 Date: Dec. 29, 2022

Related U.S. Application Data
(60) Provisional application No. 63/047,364, filed on Jul. 2, 2020.

(51) Int. Cl. C07D 211/06; A61K 45/06; C12N 5/079; A61P 25/28

(52) U.S. Cl. CPC C07D 211/06; A61K 45/06; C12N 5/0622; A61P 25/28; C12N 2501/999

(57) ABSTRACT

A method of promoting the differentiation of an oligodendrocyte precursor cell is disclosed herein. The method includes administering to the oligodendrocyte precursor cell an effective amount of a compound capable of promoting oligodendrocyte precursor cell differentiation. Also disclosed herein is a method of treating a neurodegenerative disorder, including a demyelinating disease such as multiple sclerosis, in a subject in need thereof, comprising administering to the subject a compound disclosed herein.

### CROSS-REFERENCE TO RELATED APPLICATIONS

This application claims the benefit of U.S. Provisional Patent Application No. 63/047,364, filed on Jul. 2, 2020, which is incorporated by reference herein in its entirety.

### TECHNICAL FIELD

The present invention relates generally small molecule compounds that can promote production of oligodendrocytes, and to methods for the treatment of disease in subjects where remyelination by the induction of endogenous oligodendrocyte precursor differentiation is beneficial to the subject. The present invention also relates to technology to identify such small molecule compounds.

### BACKGROUND

Multiple sclerosis (MS) is a complex neurological disease characterized by deterioration of central nervous system (CNS) myelin. Multiple sclerosis is currently the leading cause of non-traumatic neurological disability in young adults in North America. Although the cause of the disease is not clear, most patients initially experience episodes of relapses and spontaneous resolution followed by continuous neurological decline. There is no cure for MS. Current disease modifying medications are primarily anti-inflammatory in nature and while they are effective in reducing the frequency and severity of clinical attacks, they do not slow down, stop or reverse neurological disability. Spontaneous myelin repair, or remyelination, is the body's response to repair damaged myelin and protect axons, however, the efficiency of repair declines as the disease progresses.

There is a critical need to develop therapeutics to promote remyelination to prevent or reverse neurological decline. However, efforts to identify druggable targets and develop remyelination therapies have not been very successful. The myelin insulating material is composed in its majority by lipids (70% lipids), with the balance of the composition protein (30% protein). The myelin protects axons and makes possible the salutatory conduction, which speeds axonal electric impulse. Demyelination of axons in chronic MS may result in axon degeneration and neuronal cell death, but more specifically, MS destroys oligodendrocytes, the highly specialized CNS cells that generate and maintain myelin.

Oligodendrocyte precursors (PDGFRa+, NG2-proteoglycan+), the immature oligodendrocytes, are generated in ventral areas of the developing brain from a common glial progenitor, actively migrate and proliferate populating the CNS to finally differentiate to premyelinating oligodendrocytes (O4+). At this maturation point, oligodendrocytes both target and extend myelin sheaths along axons or they die. However, a population of oligodendrocyte precursors remains as resident, undifferentiated cells throughout their life supposedly to play a role as myelin recovering cells in damage or deterioration settings. Indeed, remyelination of early MS onset lesions has been reported which correlates with NG2+ oligodendrocyte progenitors detected in or around MS lesions. Nevertheless, complete, functional remyelination of MS lesions is not accomplished indicating a lack of effective maturation of resident oligodendrocyte precursors.

A less explored hypothesis of remyelination has been by either endogenous oligodendrocyte precursors or transplanted cells. Transplantation of precursor cells from diverse sources has shown promising results m terms of survival and migration of exogenous cells for long distances. Remyelination to some extent has also been reported in several experimental models of demyelination after transplantation of neural precursors and stem cells. Yet, remyelination of multiple demyelinated areas by transplanted cells would require multiple transplantation loci, which in practice limits the effectiveness and clinical applicability of this approach.

Promoting remyelination by inducing differentiation of endogenous oligodendrocyte progenitors can stimulate and enhance intrinsic, natural remyelination. Therefore, there is a need for compounds and therapeutic methods capable inducing endogenous oligodendrocyte precursor differentiation.

### SUMMARY

The present invention relates generally to compounds and methods for oligodendrocyte precursor cell differentiation. The present invention also relates to methods for the treatment of disease in subjects where remyelination by the induction of endogenous oligodendrocyte precursor differentiation is beneficial to the subject.

To address the present critical need a novel technology has been developed to identify small molecule compounds that can promote production of oligodendrocytes, the myelin-producing cells. The unique technological innovations include:

1) an optimized method to produce primary culture of highly pure mouse oligodendrocyte progenitor cells (OPC); and

2) an algorithm to identify and quantify oligodendrocytes in 96-well format.

Using this medium throughput, primary cell-based phenotypic screening approach, compounds are rapidly identified that can promote oligodendrocyte generation for development as remyelination therapeutics.

The present invention further relates to a method of promoting oligodendrocyte precursor cell differentiation, comprising administering to one or more oligodendrocyte precursor cells an effective amount of a compound of formula (IV):

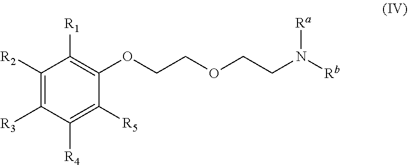

or a pharmaceutically acceptable salt thereof, wherein: X is selected from N and CH; R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R^{a} and R^{b} are each independently selected from hydrogen and alkyl, or R^{a} and R^{b} are taken together with the nitrogen atom to which they are attached to form an optionally substituted heterocyclyl. In some embodiments, R₁, R₂, R₃, R₄, and R₅ are each independently selected from the group consisting of hydrogen, alkyl, and halo.

In some embodiments, the compound of formula (IV) is a compound selected from the group consisting of:

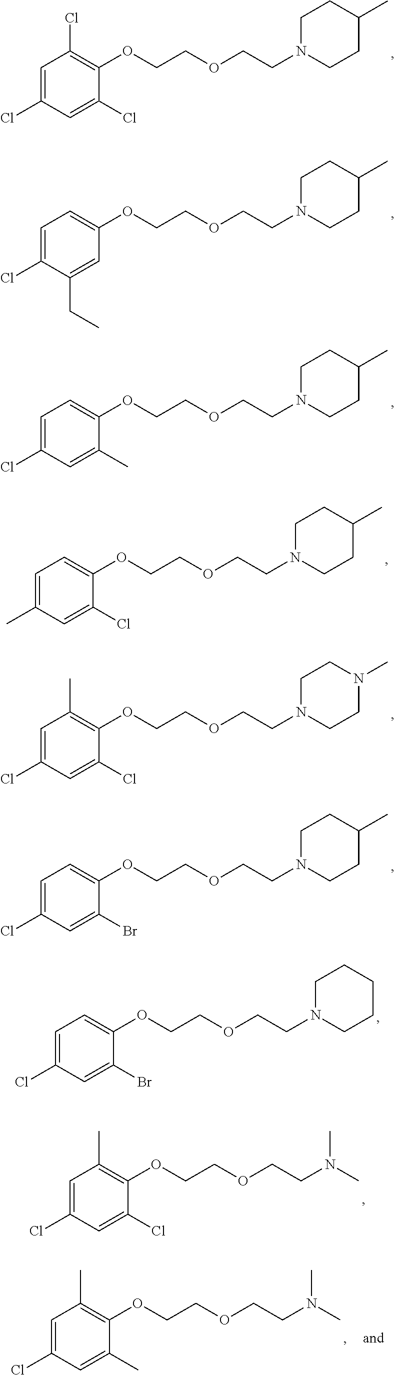

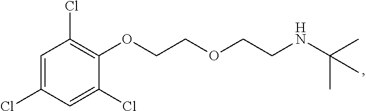 [SMILES: CC(C)(C)NCCOCCOc1c(Cl)cc(Cl)cc1Cl]

and pharmaceutically acceptable salts thereof.

The present invention further relates to a method of promoting oligodendrocyte precursor cell differentiation, comprising administering to one or more oligodendrocyte precursor cells an effective amount of a compound of formula (I):

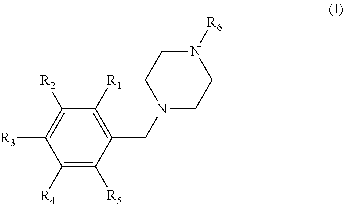

or a pharmaceutically acceptable salt thereof, wherein: R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R⁶ is a group of formula —(CH₂)ₙ-A, wherein n is 0 or 1, and A is selected from aryl, heteroaryl, cycloalkyl, cycloalkenyl, and heterocyclyl, wherein the aryl, heteroaryl, cycloalkyl, cycloalkenyl, and heterocyclyl are optionally substituted with 1, 2, or 3 substituents.

The present invention also relates to method of promoting oligodendrocyte precursor cell differentiation comprising administering to one or more oligodendrocyte precursor cells an effective amount of a compound selected from the group consisting of:

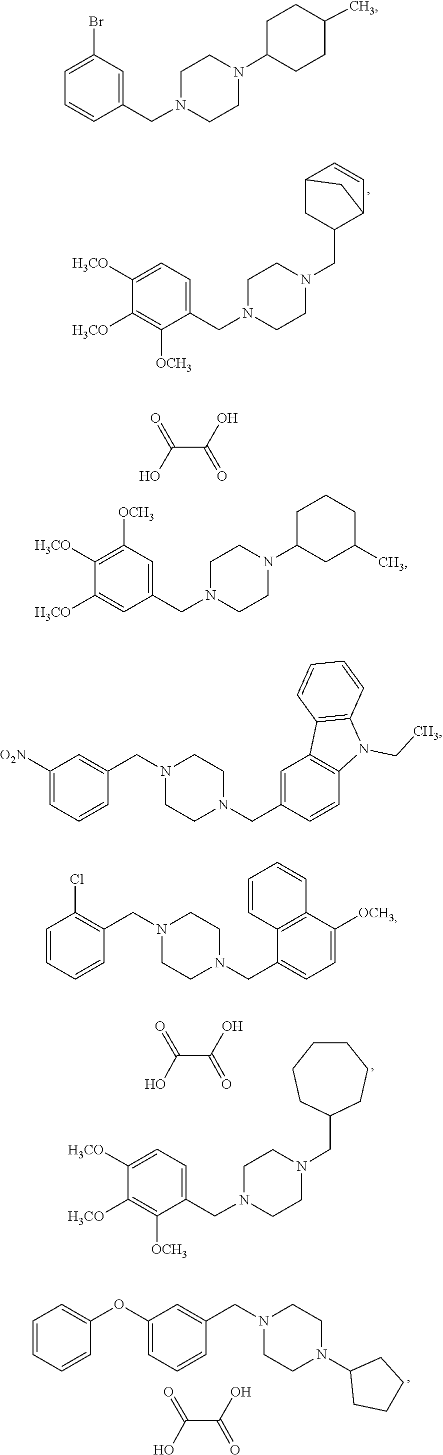

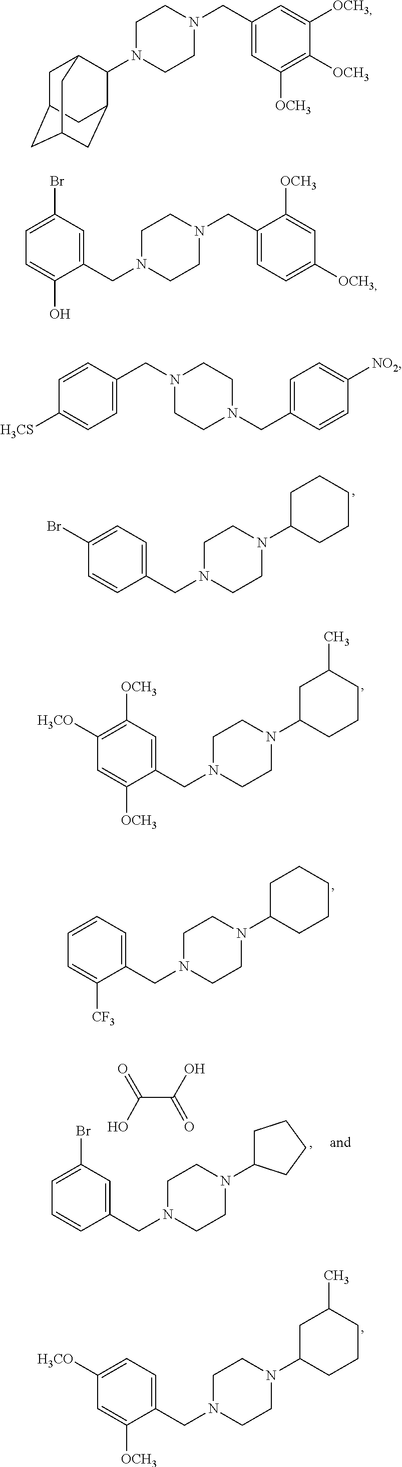

and pharmaceutically acceptable salts thereof.

The present invention further relates to a method of promoting oligodendrocyte precursor cell differentiation, comprising administering to one or more oligodendrocyte precursor cells an effective amount of a compound of formula (II):

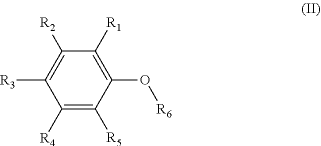

or a pharmaceutically acceptable salt thereof, wherein: R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; wherein R₁ and R₂, R₂ and R₃, R₃ and R₄, or R₄ and R₅ are optionally taken together with the carbon atoms to which they are attached to form a ring; and R⁶ is a group of formula —(CH₂)ₙ—(C(O))ₘ—NR^{a}R^{b}, wherein: m is 0 or 1; n is 1, 2, 3, 4, 5, or 6; and R^{a} and R^{b} are each independently selected from alkyl, or R^{a} and R^{b} are taken together with the nitrogen atom to which they are attached to form an optionally substituted heterocyclyl.

The present invention also relates to method of promoting oligodendrocyte precursor cell differentiation comprising administering to one or more oligodendrocyte precursor cells an effective amount of a compound selected from the group consisting of:

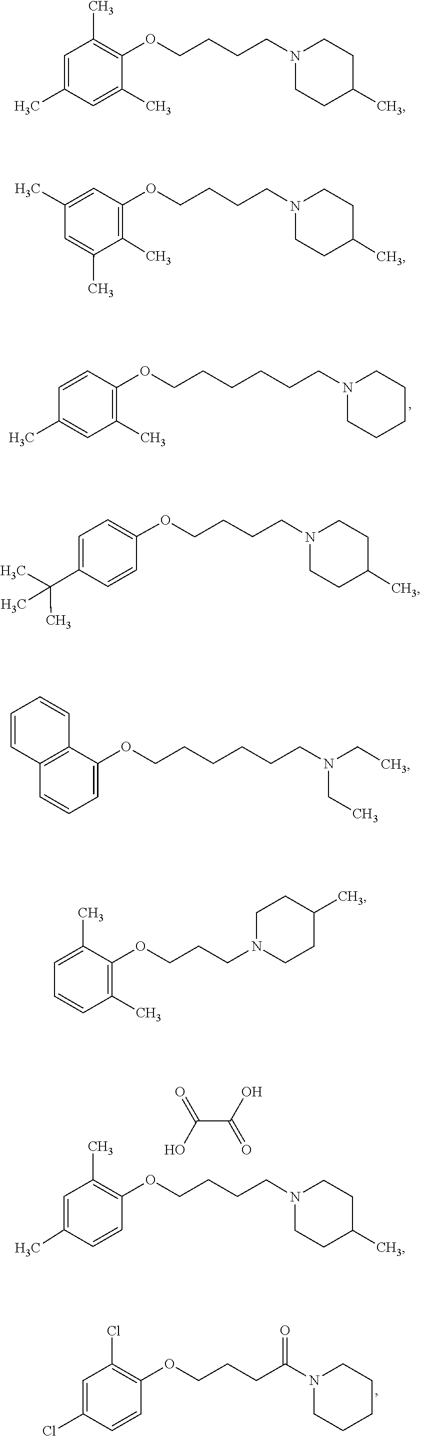

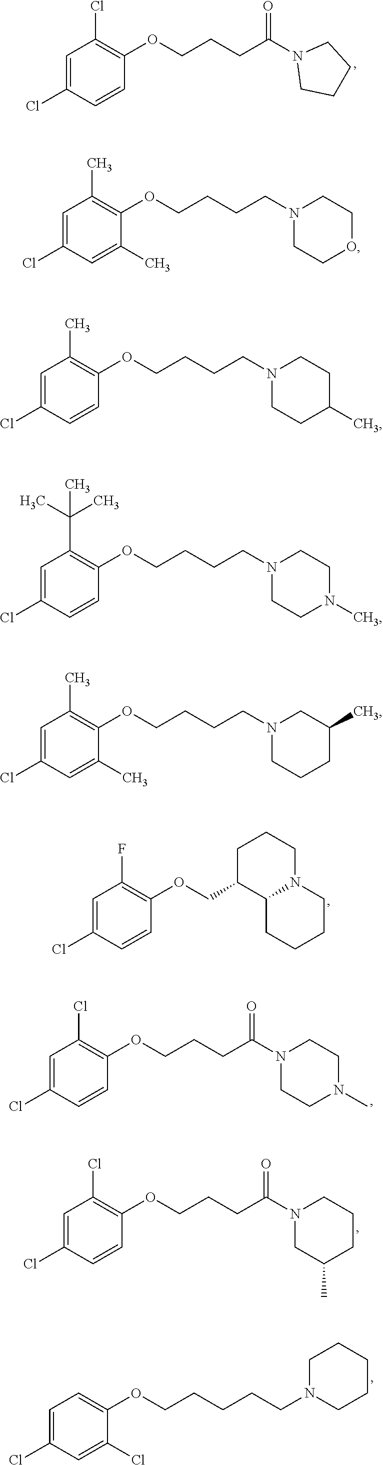

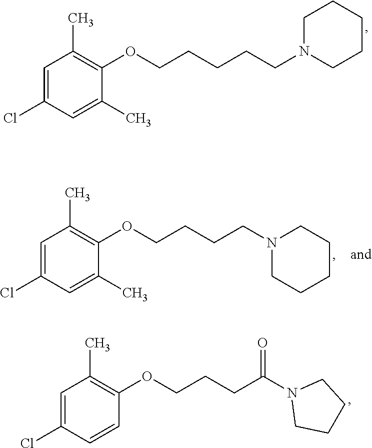 [SMILES: Cc1cc(Cl)cc(C)c1OCCCCCN1CCCCC1 ; Cc1cc(Cl)cc(C)c1OCCCCN1CCCCC1 ; Cc1cc(Cl)ccc1OCCCC(=O)N1CCCC1]

and pharmaceutically acceptable salts thereof.

The present invention further relates to a method of promoting oligodendrocyte precursor cell differentiation, comprising administering to one or more oligodendrocyte precursor cells an effective amount of a compound of formula (III):

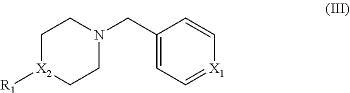

or a pharmaceutically acceptable salt thereof, wherein X₁ and X₂ are each independently selected from CH and N, and R₁ is selected from the group consisting of cycloalkyl and heterocyclyl, each of which is optionally substituted with 1 or 2 substituents.

The present invention also relates to method of promoting oligodendrocyte precursor cell differentiation comprising administering to one or more oligodendrocyte precursor cells an effective amount of a compound selected from the group consisting of:

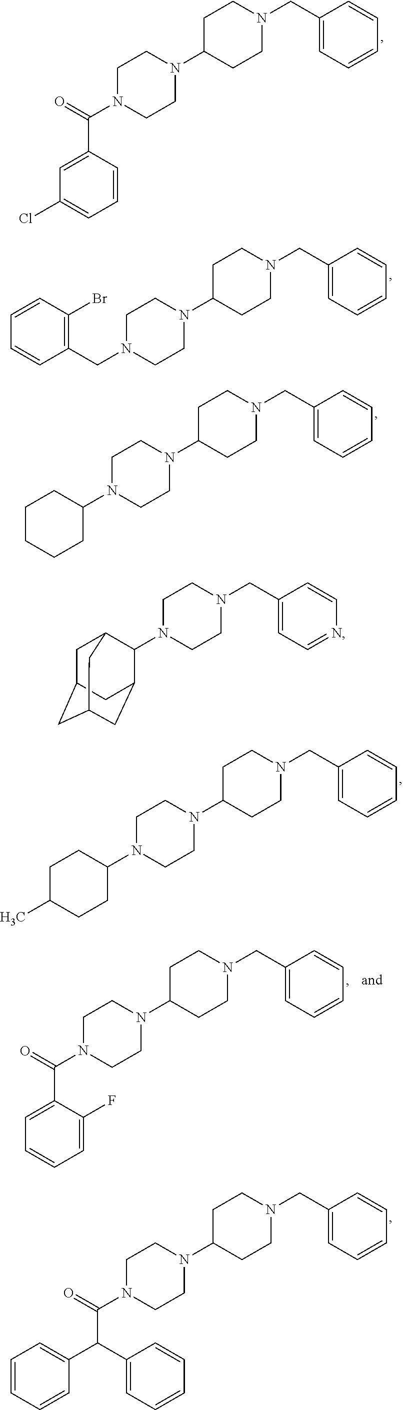

and pharmaceutically acceptable salts thereof.

The present invention also relates to method of promoting oligodendrocyte precursor cell differentiation comprising administering to one or more oligodendrocyte precursor cells an effective amount of a compound selected from the group consisting of:

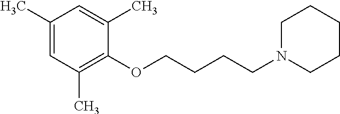 [SMILES: Cc1cc(C)c(OCCCCN2CCCCC2)c(C)c1]

and pharmaceutically acceptable salts thereof.

The present invention also relates to method of promoting oligodendrocyte precursor cell differentiation comprising administering to one or more oligodendrocyte precursor cells an effective amount of a compound selected from the group consisting of:

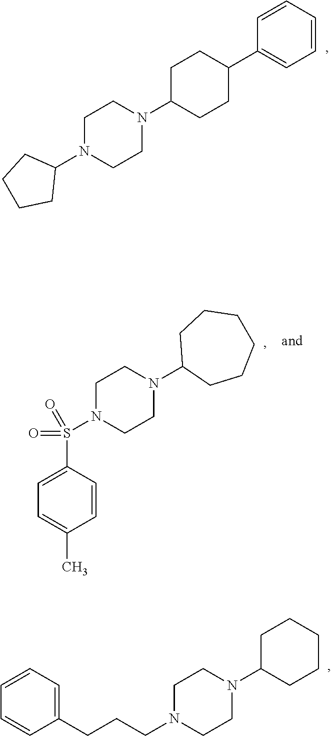 [SMILES: c1ccc(C2CCC(N3CCN(C4CCCC4)CC3)CC2)cc1 ; Cc1ccc(S(=O)(=O)N2CCN(C3CCCCCC3)CC2)cc1 ; c1ccc(CCCN2CCN(C3CCCCC3)CC2)cc1]

and pharmaceutically acceptable salts thereof.

The present invention also relates to method of promoting oligodendrocyte precursor cell differentiation comprising administering to one or more oligodendrocyte precursor cells an effective amount of a compound selected from the group consisting of:

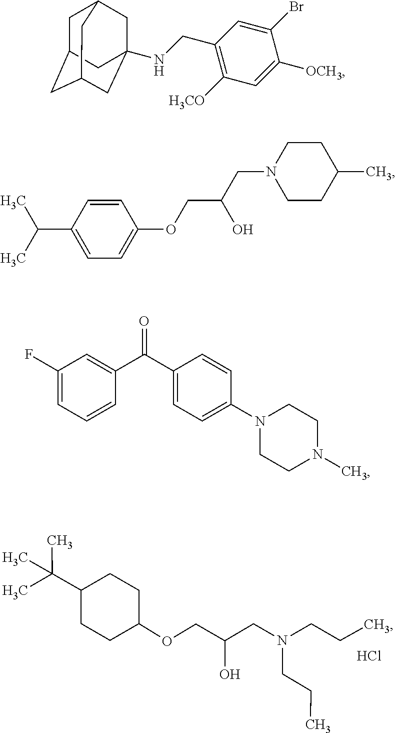 [SMILES: COc1cc(OC)c(CNC23CC4CC(CC(C4)C2)C3)cc1Br ; CC1CCN(CC(O)COc2ccc(C(C)C)cc2)CC1 ; CN1CCN(c2ccc(C(=O)c3cccc(F)c3)cc2)CC1 ; CCCN(CCC)CC(O)COC1CCC(C(C)(C)C)CC1 ; Cl]

and pharmaceutically acceptable salts thereof.

The present invention further relates to a method of promoting oligodendrocyte precursor cell differentiation, comprising administering to one or more oligodendrocyte precursor cells an effective amount of a compound selected from:

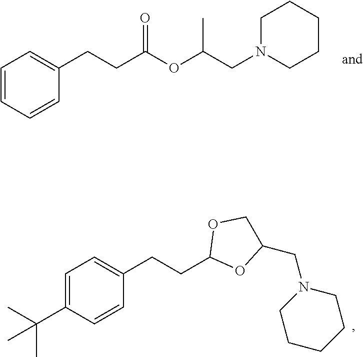 [SMILES: CC(CN1CCCCC1)OC(=O)CCc1ccccc1 ; CC(C)(C)c1ccc(CCC2OCC(CN3CCCCC3)O2)cc1]

and pharmaceutically acceptable salts thereof.

The present invention also relates to a method of treating a neurodegenerative disorder, including a demyelinating disease such as multiple sclerosis, in a subject in need thereof, comprising administering to the subject a compound disclosed herein (e.g., a compound of formula (I), (II), (III), or (IV), or any compound disclosed herein).

Further features of the invention will become apparent in the course of the following description.

### BRIEF DESCRIPTION OF THE DRAWINGS

FIGS. 1A-1B show structures and data for “hit” compounds identified in screening. FIG. 1A: chemical structures and properties of “hit” compounds; FIG. 1B: Western blot quantification of PLP protein levels in response to several different small molecule hit compounds over three days. DMSO: negative control; T3: positive control; the Y-axis is normalized densitometry and each line represents one hit compound.

FIG. 2 shows a comparison of compound CN045 with two T3 and two other repurposed drugs, benzotropine and clemastine, in an oligodendrocyte progenitor cell (OPC) assay.

FIGS. 3A-3D show data demonstrating that compound CN045 promotes OPC differentiation with good potency in mice. FIG. 3A: CN045-activated expression of oligodendrocyte markers in the same cells, green: EGFP driven by the PLP promoter, red in top panel: PLP protein, red in bottom panel: MBP protein; FIG. 3B: dose-response and EC50, where CN045 produced ˜4-fold increases in the percentage of oligodendrocytes compared with DMSO controls; FIG. 3C: plasma concentration over time for CN045 following intraperitoneal injection at 10 mg/kg body weight; plasma samples were taken at 1, 2, 4, 8, and 24 hr after injection and concentration determined by LC/MS/MS, n=3 mice each group, error bar: standard deviation; FIG. 3D: brain penetration of CN045; the brain/plasma ratio indicates good brain penetration.

FIGS. 4A-4I show data demonstrating that CN045 promotes remyelination in a cuprizone mouse model of demyelination/remyelination. Compound and vehicle were i.p. injected daily for 6 weeks, following 12 weeks of cuprizone/rapamycin demyelination. FIGS. 4A-4B: PLP immunostaining of cortex; FIGS. 4C-4D: PLP immunostaining of hippocampus; FIGS. 4E-4F: myelinated axons in corpus callosum; FIGS. 4G-4I: quantification of remyelination in cortex, hippocampus, and corpus callosum. **, p<0.01; *, p<0.05; Student's t-test. Error bar: standard deviation. N=12 mice for each group.

### DETAILED DESCRIPTION

In referring to the description of the preferred embodiments as described herein, usefulness may be found in understanding the goal of the developmental research that led to these inventions was to identify and evaluate potential candidate compounds from a library of over 20,000 small molecules. Using the screening platform developed herein and through voluminous trial and error, identification of 390 compounds were characterized as highly effective positives. Further validation revealed 43 of these compounds with sub-micromolar EC₅₀. Based on evaluation of key parameters including EC₅₀, toxicity and differentiation profile, identification of lead compounds were accomplished in which an EC₅₀ of 30 nM exists. While not intended to be definitively authoritative of the present embodiments, preliminary analysis suggested that the compound works upstream of AKT to regulate oligodendrocyte differentiation. The primary embodiments are thereby thought to be highly appealing drug candidate in that its variants have low cytotoxicity, good aqueous solubility and is capable of crossing the blood-brain barrier.

Again, by way of disclosure and not intended to be definitively authoritative of the present embodiments, it is currently thought that compounds of the primary embodiment may promote oligodendrocyte production and remyelination in demyelinated brains.

Further for purposes of the present application and for convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as” and “e.g.” as used herein are non-limiting and are for illustrative purposes only. “Including” and “including but not limited to” are used interchangeably.

The term “or” as used herein should be understood to mean “and/or” unless the context clearly indicates otherwise.

In the present specification, the structural formula of the compound represents a certain isomer for convenience in some cases, but the present invention includes all isomers such as geometrical isomer, optical isomer based on an asymmetrical carbon, stereoisomer, tautomer and the like which occur structurally and an isomer mixture and is not limited to the description of the formula for convenience, and may be any one of isomer or a mixture.

Therefore, an asymmetrical carbon atom may be present in the molecule and an optically active compound and a racemic compound may be present in the present compound, but the present invention is not limited to these and includes any one. In addition, a crystal polymorphism may be present but is not limiting, but any crystal form may be single or a crystal form mixture, or an anhydride or hydrate. Further, so-called metabolite, which is produced by degradation of the present compound in vivo, is included in the scope of the present invention.

It will be noted that the structure of some of the compounds of the invention include asymmetric (chiral) carbon atoms. It is to be understood accordingly that the isomers arising from such asymmetry are included within the scope of the invention, unless indicated otherwise. Such isomers can be obtained in substantially pure form by classical separation techniques and by stereochemically controlled synthesis. The compounds of this invention may exist in stereoisomeric form, therefore can be produced as individual stereoisomers or as mixtures.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers” and stereoisomers that are non-superimposable mirror images are termed “enantiomers,” or sometimes optical isomers. A carbon atom bonded to four non-identical substituents is termed a “chiral center.”

“Chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture.” A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Int. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric Isomers” means diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however, as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate, and when the solvent is alcohol, the solvate formed is an alcoholate. Hydrates are formed by the combination of one or more molecules of water with one of the substances in which the water retains its molecular state as H₂O, such combination being able to form one or more hydrate.

“Tautomers” refers to compounds whose structures differ markedly in arrangement of atoms, but which exist in easy and rapid equilibrium. It is to be understood that certain compounds disclosed herein may be depicted as different tautomers. It should also be understood that when compounds have tautomeric forms, all tautomeric forms are intended to be within the scope of the invention, and the naming of the compounds does not exclude any tautomer form.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonamides, tetrazoles, sulfonates, and phosphonates. See, e.g., Patani and LaVoie, Chem. Rev. 96, 3147-3176 (1996).

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject which may be predisposed to the disease, disorder and/or condition but has not yet been diagnosed as having it. Preventing a condition related to a disease includes stopping the condition from occurring after the disease has been diagnosed but before the condition has been diagnosed.

A “pharmaceutical composition” is a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of a compound of this invention include powders, sprays, ointments, pastes, creams, lotions, gels, solutions, patches and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, com oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds of the invention are capable of further forming salts. All of these forms are also contemplated within the scope of the claimed invention.

“Pharmaceutically acceptable salt” of a compound means a salt that is pharmaceutically acceptable and that possesses the desired pharmacological activity of the parent compound. For example, the salt can be an acid addition salt. One embodiment of an acid addition salt is a hydrochloride salt. Another embodiment of an acid addition salt is an oxalate salt. The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the present invention. It should be understood that all references to pharmaceutically acceptable salts include solvent addition forms (solvates) or crystal forms (polymorphs) as defined herein, of the same salt.

The compounds of the present invention can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds of the present invention can also be prepared as prodrugs, for example pharmaceutically acceptable prodrugs. The terms “pro-drug” and “prodrug” are used interchangeably herein and refer to any compound, which releases an active parent drug m vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy, or carbonyl group is bonded to any group that may be cleaved in vivo to form a free hydroxy!, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, ester groups (e.g. ethyl esters, morpholinocthanol esters) of carboxy I functional groups, N-acyl derivatives (e.g. N-acetyl) N-Mannich bases, Schiff bases and enaminones of amino functional groups, oximes, acetals, ketals and enol esters of ketone and aldehyde functional groups in compounds of Formula I, and the like, See Bundegaard, H. “Design of Prodrugs” pi-92, Elsevier, New York-Oxford (1985).

“Protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2.sup.nd ed. 1991); Harrison and Harrison et al., Compendium of Synthetic Organic Methods, Vols. 1-8 (John Wiley and Sons, 1971-1996); and Kocienski, Protecting Groups, (Verlag, 3.sup.rd ed. 2003).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent,” “drug,” “medicament,” and “bioactive substance” are art-recognized and include molecules and other agents that are biologically, physiologically, or pharmacologically active substances that act locally or systemically in a patient or subject to treat a disease or condition, such as macular degeneration or other forms of retinal disease whose etiology involves aberrant clearance of all trans-retinal. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “effective amount” are art-recognized terms. In certain embodiments, “therapeutically effective amount” or “effective amount,” in terms of each foregoing methods, is the amount of the compounds described herein effective to induce or promote differentiation of at least one oligodendrocyte precursor.

The term “ED50” is art-recognized. In certain embodiments, ED50 means the dose of a drug, which produces 50% of its maximum response or effect, or alternatively, the dose which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drag, defined as LD50/ED50.

With respect to any chemical compounds, the present invention is intended to include all isotopes of atoms occurring in the present compounds. Isotopes include those atoms having the same atomic number but different mass numbers. Examples of isotopes suitable for inclusion in the compounds of the disclosure are hydrogen, carbon, nitrogen, oxygen, phosphorus, sulfur, fluorine, and chlorine, such as ²H, ³H, ¹³C, ¹⁴C, ¹⁵N, ¹⁸O, ³¹P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in the compounds are ¹¹C, ¹³N, ¹⁵O, and ¹⁸F. Isotopically-labeled compounds can generally be prepared by conventional techniques known to those skilled in the art or by processes analogous to those described herein using an appropriate isotopically-labeled reagent in place of a non-isotopically-labeled reagent.

The chemical compounds described herein can have asymmetric centers. Compounds of the present invention containing an asymmetrically substituted atom can be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and can be isolated as a mixture of isomers or as separated isomeric forms. All chiral, diastereomeric, racemic, and geometric isomeric forms of a structure are intended, unless the specific stereochemistry or isomeric form is specifically indicated. All processes used to prepare compounds of the present invention and intermediates made therein are, where appropriate, considered to be part of the present invention. All tautomers of shown or described compounds are also, where appropriate, considered to be part of the present invention.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

The phrase “having the formula” or “having the structure” is not intended to be limiting and is used in the same way that the term “comprising” is commonly used.

“Effective amount,” in terms of each foregoing methods, is the amount of the compounds described herein effective to induce or promote differentiation of at least one oligodendrocyte precursor.

The term “alkyl” refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups, such as cyclopentyl, cyclohexyl, and the like. Generally, although again not necessarily, alkyl groups herein contain 1 to about 18 carbon atoms, preferably 1 to about 12 carbon atoms.

“Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail intra. If not otherwise indicated, the term “alkyl” includes linear, branched, cyclic, unsubstituted, substituted, and/or heteroatom-containing alkyl, respectively.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, cyclopentenyl, cyclohexenyl, cyctooctenyl, and the like. Generally, although again not necessarily, alkenyl groups can contain 2 to about 18 carbon atoms, and more particularly 2 to 12 carbon atoms. The term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl or heterocycloalkenyl (e.g., heterocyclohexenyl) in which at least one carbon atom is replaced with a heteroatom. If not otherwise indicated, the terms “alkenyl” includes linear, branched, cyclic, unsubstituted, substituted, and/or heteroatom-containing alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups can contain 2 to about 18 carbon atoms, and more particularly can contain 2 to 12 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the term “alkynyl” include linear, branched unsubstituted, substituted, and/or heteroatom-containing alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc.

The term “amino” refers to a group —NR₂, wherein each R is independently selected from hydrogen and alkyl (as defined herein). Accordingly, when the term “amino” is used herein, the term encompasses an —NH₂ group, an alkylamino group (e.g., —NHCH₃), and a dialkylamino group (—N(CH₃)₂).

The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 20 carbon atoms, and, for example, can contain 5 to 14 carbon atoms. Examples aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatics.

The term “aryloxy” as used herein refers to an aryl group bound through a single, terminal ether linkage, wherein “aryl” is as defined above. An “aryloxy” group may be represented as —O-aryl where aryl is as defined above. Aryloxy groups can contain 5 to 20 carbon atoms, and can contain, for example, 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2, 4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Examples of aralkyl groups contain 6 to 24 carbon atoms, and particularly preferred aralkyl groups contain 6 to 16 carbon atoms.

The term “acyl” refers to a group —C(═O)R, where R is selected from the group consisting of alkyl, alkenyl, alkynyl, aryl, arylalkyl, cycloalkyl, cycloalkylalkyl, heteroaryl, heteroarylalkyl, heterocyclyl, heterocyclylalkyl, and heteroalkyl.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, fluoro, or iodo substituent.

The term “haloalkyl” refers to an alkyl group, as defined herein, in which at least one hydrogen atom (e.g., one, two, three, four, five, six, seven or eight hydrogen atoms) is replaced by a halogen. Representative examples of haloalkyl include, but are not limited to, fluoromethyl, difluoromethyl, trifluoromethyl, 2,2,2-trifluoroethyl, and 3,3,3-trifluoropropyl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen, sulfur. Similarly, the term “heteroalkyl” refers to an alkyl substituent that is heteroatom-containing, the term “heterocyclyl” or “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, I, 2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. If a particular group permits, it may be further substituted with one or more additional functional groups or with one or more hydrocarbyl moieties. Analogously, the above-mentioned hydrocarbyl moieties may be further substituted with one or more functional groups or additional hydrocarbyl moieties.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

The present invention relates to compounds and methods for promoting differentiation of oligodendrocyte precursors. Compounds in accordance with the invention can be used in the treatment of neurodegenerative disorders, such as multiple sclerosis, to induce and or promote differentiation of oligodendrocyte precursor cells. The term “oligodendrocyte precursor cells”¹ as used herein refers immature oligodendrocyte cells. Oligodendrocyte precursor cells can be identified by the expression of a number of surface antigens. For example, the surface antigens known as platelet-derived growth factor-alpha receptor subunit (PDGFRα), NG2 chondroitin sulfate proteoglycan, and ganglioside GD3, are commonly used to identify oligodendrocyte precursor cells.

Immature oligodendrocyte precursors are generated in ventral areas of the developing brain from a common glial progenitor. The immature cells actively migrate and proliferate populating the CNS to finally differentiate to premyelinating oligodendrocytes (O4+). Oligodendrocyte precursor differentiation and maturation is characterized by an extension of multiple processes, increase in cell body size and formation of myelin.

In one aspect of the present invention, the compounds in accordance with the present invention are identified using a high-throughput small molecule screen that is biased to identify compounds that have both a high potency and low toxicity in mammal subjects and are able to promote oligodendrocyte precursor differentiation. The term \hat{ }small molecule” as used herein refers to biologically active organic compounds of low molecular weight (e.g. <500 kDa) which may cross biological membranes and modulate intracellular processes.

Briefly, the high-throughput small molecule screen included a primary screening where small drug-like organic compounds (250-550 kDa) are added to cells seeded on a 96-well plate an incubated. The cells are then visually screened for oligodendrocyte precursor morphology changes. In a secondary screening, differentiation induced by selected compounds was further validated by fluorescence microscopy. Increased fluorescence in treated oligodendrocyte cells generated from a PIp-EGFP transgenic mouse was indicative of cell maturation. Further oligodendrocyte precursor maturation in response to selected compounds was assessed by induction of myelin protein expression as determined by immunocytochemistry and western blot.

Examples of compounds identified by the high-throughput small molecule screen that can be used to promote oligodendrocyte precursor differentiation include compounds of formulae (I), (II), (III), and (IV), and other compounds disclosed herein.

Accordingly, the present invention further relates to a method of promoting oligodendrocyte precursor cell differentiation, comprising administering to one or more oligodendrocyte precursor cells an effective amount of a compound of formula (IV):

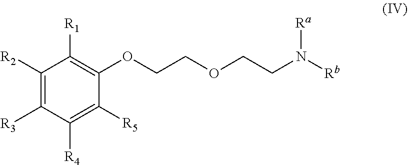

or a pharmaceutically acceptable salt thereof, wherein: X is selected from N and CH; R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R^{a} and R^{b} are each independently selected from hydrogen and alkyl, or R^{a} and R^{b} are taken together with the nitrogen atom to which they are attached to form an optionally substituted heterocyclyl.

In some embodiments, R₁, R₂, R₃, R₄, and R₅ are each independently selected from the group consisting of hydrogen, alkyl (e.g., C₁-C₄ alkyl), and halo (e.g., chloro or bromo). In some embodiments, R₂ and R₄ are hydrogen, and R₁, R₃, and R₅ are each independently selected from alkyl (e.g., C₁-C₄ alkyl, such as methyl) and halo (e.g., chloro or bromo). In some embodiments, R₁, R₂ and R₅ are hydrogen, and R₃ and R₄ are each independently selected from alkyl (e.g., C₁-C₄ alkyl, such as methyl or ethyl) and halo (e.g., chloro or bromo).

In some embodiments, R^{a} and R^{b} are each independently selected from hydrogen and C₁-C₄ alkyl (e.g., methyl or tert-butyl), or R^{a} and R^{b} are taken together with the nitrogen atom to which they are attached to form an optionally substituted six-membered heterocyclyl (e.g., piperidinyl, piperazinyl, or morpholino).

In some embodiments, the compound of formula (IV) is a compound of formula (IVa):

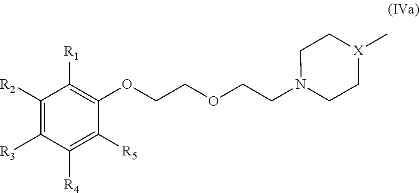

or a pharmaceutically acceptable salt thereof, wherein: X is selected from N and CH; and R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso.

Compounds of formula (IV) include compound CN045, and may be referred to generally herein as the “CN045 Series.”

In some embodiments, the compound of formula (IV) is selected from the group consisting of:

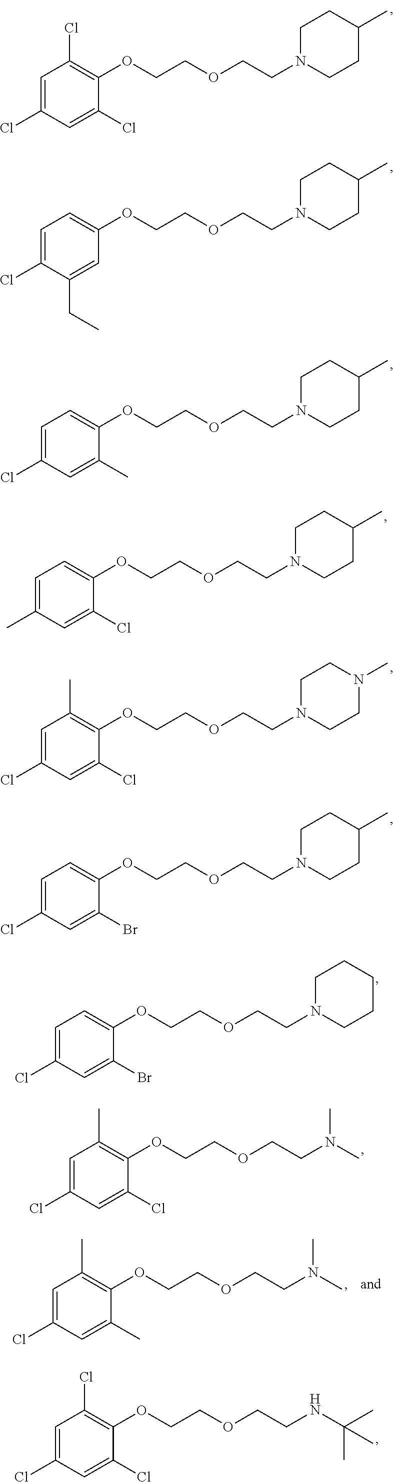

and pharmaceutically acceptable salts thereof.

The present invention further relates to a method of promoting oligodendrocyte precursor cell differentiation, comprising administering to one or more oligodendrocyte precursor cells an effective amount of a compound of formula (I):

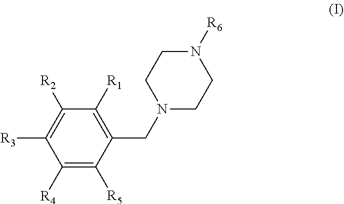

or a pharmaceutically acceptable salt thereof, wherein R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R⁶ is a group of formula —(CH₂)ₙ-A, wherein n is 0 or 1, and A is selected from aryl, heteroaryl, cycloalkyl, cycloalkenyl, and heterocyclyl, wherein the aryl, heteroaryl, cycloalkyl, cycloalkenyl, and heterocyclyl are optionally substituted with 1, 2, or 3 substituents.

In some embodiments, R₁, R₂, R₃, R₄, and R₅ are each independently selected from the group consisting of hydrogen, alkyl, halo (e.g., chloro or bromo), hydroxy, alkoxy (e.g., methoxy), aryloxy (e.g., phenoxy), nitro, haloalkyl (e.g., trifluoromethyl), and thioalkyl (e.g., methylthio). In some embodiments, one, two, or three of R₁, R₂, R₃, R₄, and R₅ are independently selected from the group consisting of halo, hydroxy, alkoxy, aryloxy, nitro, haloalkyl, and thioalkyl, and the rest are hydrogen.

In some embodiments, R₆ is a group of formula —(CH₂)ₙ-A, wherein n is 0 or 1, and A is selected from aryl (e.g., phenyl or naphthyl), heteroaryl (e.g., carbazolyl), and C₃-C₁₀ cycloalkyl (e.g., cyclopentyl, cyclohexyl, cycloheptyl, or adamantyl), and cycloalkenyl (e.g., bicyclo[2.2.1]heptenyl), each of which is optionally substituted with 1 or 2 substituents independently selected from C₁-C₄ alkyl (e.g., methyl or ethyl), C₁-C₄ alkoxy (e.g., methoxy), halo (e.g., chloro or bromo), C₁-C₄ thioalkyl (e.g., methylthio), hydroxy, and nitro.

Compounds of formula (I) include compound CN007, and may be referred to generally herein as the “CN007 Series.”

Examples of compounds having formula (I) that may be used to promote differentiation of oligodendrocyte precursors include:

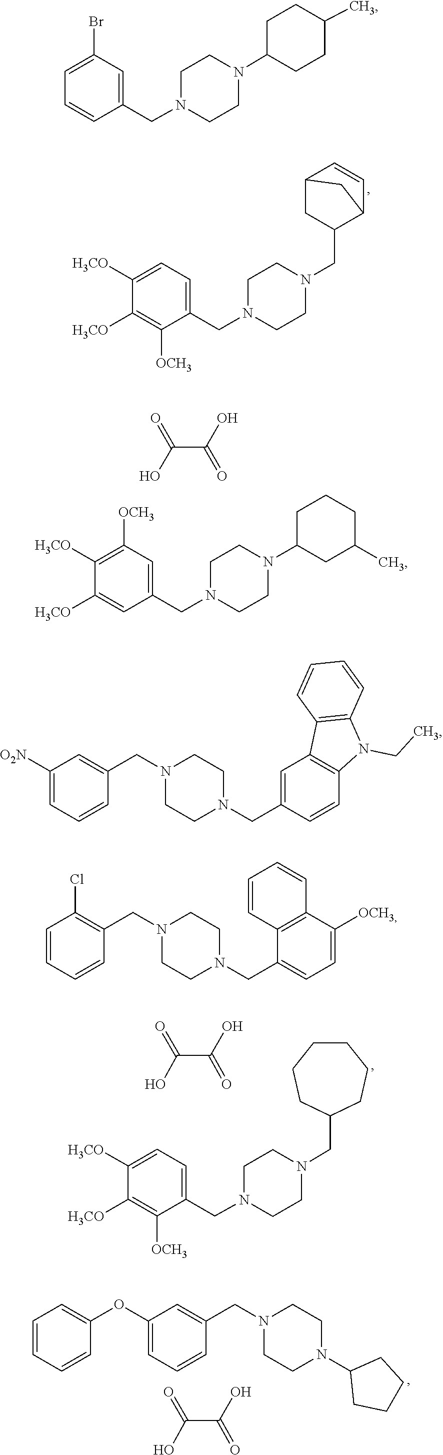

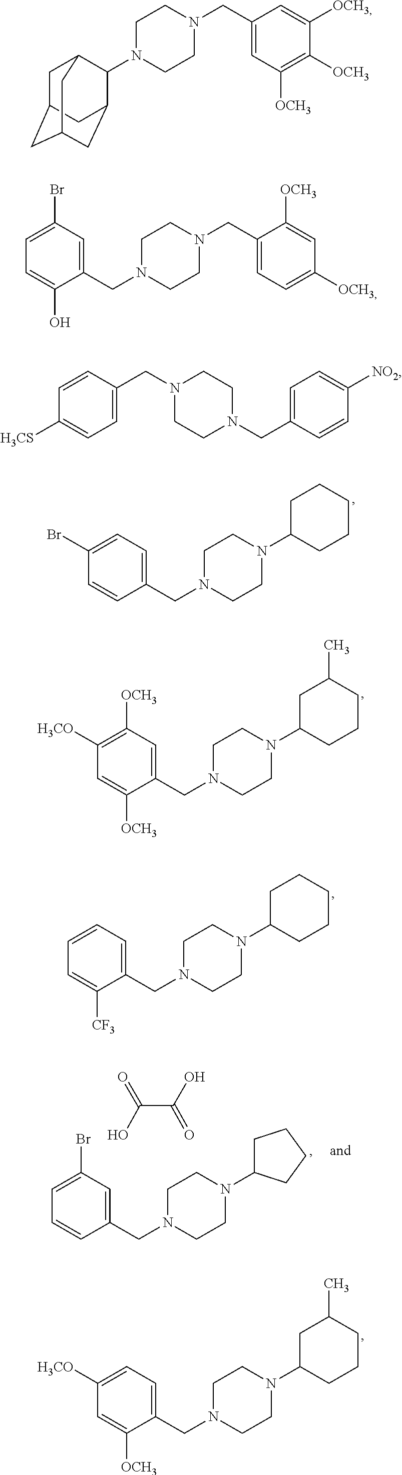

and pharmaceutically acceptable salts thereof.

The present invention further relates to a method of promoting oligodendrocyte precursor cell differentiation, comprising administering to one or more oligodendrocyte precursor cells an effective amount of a compound of formula (II):

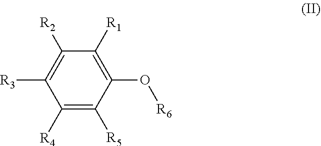

or a pharmaceutically acceptable salt thereof, wherein R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; wherein R₁ and R₂, R₂ and R₃, R₃ and R₄, or R₄ and R₅ are optionally taken together with the carbon atoms to which they are attached to form a ring (e.g., an aryl); wherein R₁ and R₂, R₂ and R₃, R₃ and R₄, or R₄ and R₅ are optionally taken together with the carbon atoms to which they are attached to form a ring; and R⁶ is a group of formula —(CH₂)ₙ—(C(O))ₘ—NR^{a}R^{b}, wherein: m is 0 or 1; n is 1, 2, 3, 4, 5, or 6; and R^{a} and R^{b} are each independently selected from alkyl, or R^{a} and R^{b} are taken together with the nitrogen atom to which they are attached to form an optionally substituted heterocyclyl.

In some embodiments, R₁, R₂, R₃, R₄, and R₅ are each independently selected from hydrogen, alkyl (e.g., methyl or tert-butyl), and halo (e.g., fluoro or chloro). In some embodiments, one, two, or three of R₁, R₂, R₃, R₄, and R₅ are alkyl (e.g., methyl or tert-butyl) or halo (e.g., fluoro or chloro), and the others are hydrogen. In some embodiments, R₁ and R₂ are taken together with the carbon atoms to which they are attached to form an aryl ring.

In some embodiments, R₆ is a group of formula —(CH₂)ₙ—(C(O))ₘ—NR^{a}R^{b}, wherein: m is 0 or 1; n is 1, 2, 3, 4, 5, or 6; and R^{a} and R^{b} are each independently selected from C₁-C₄ alkyl (e.g., methyl), or R^{a} and R^{b} are taken together with the nitrogen atom to which they are attached to form an optionally substituted 5- or 6-membered monocyclic heterocyclyl (e.g., pyrrolidinyl, piperidinyl, piperazinyl, or morpholino), wherein the heterocyclyl is optionally substituted with one C₁-C₄ alkyl group (e.g., methyl). In some embodiments, m is 0. In some embodiments, m is 1. In some embodiments, n is 2. In some embodiments, n is 3. In some embodiments, n is 4. In some embodiments, n is 5. In some embodiments, n is 6.

Examples of compounds having formula (II) that may be used to promote differentiation of oligodendrocyte precursors include:

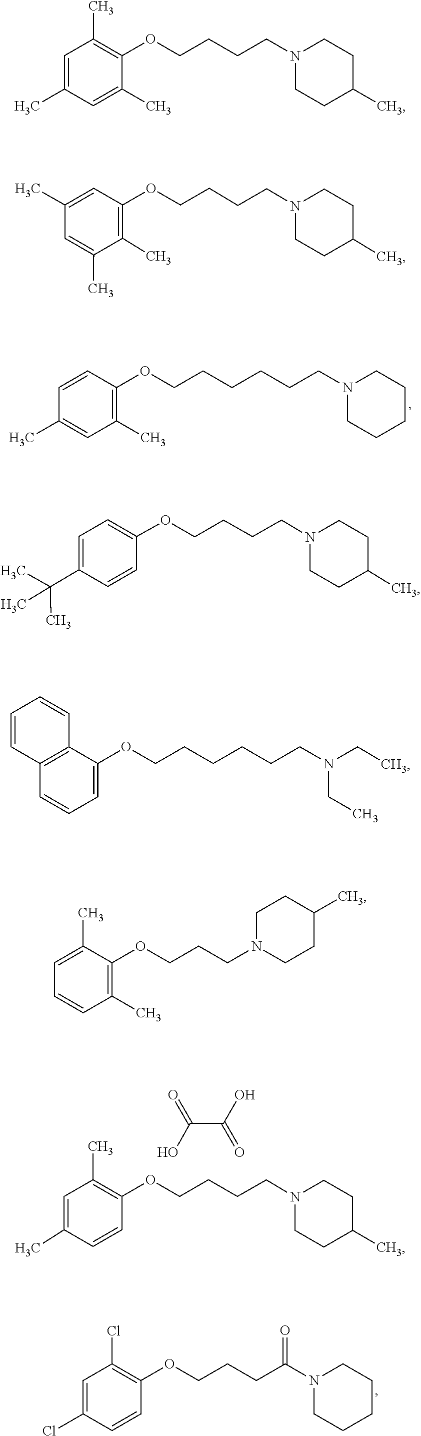

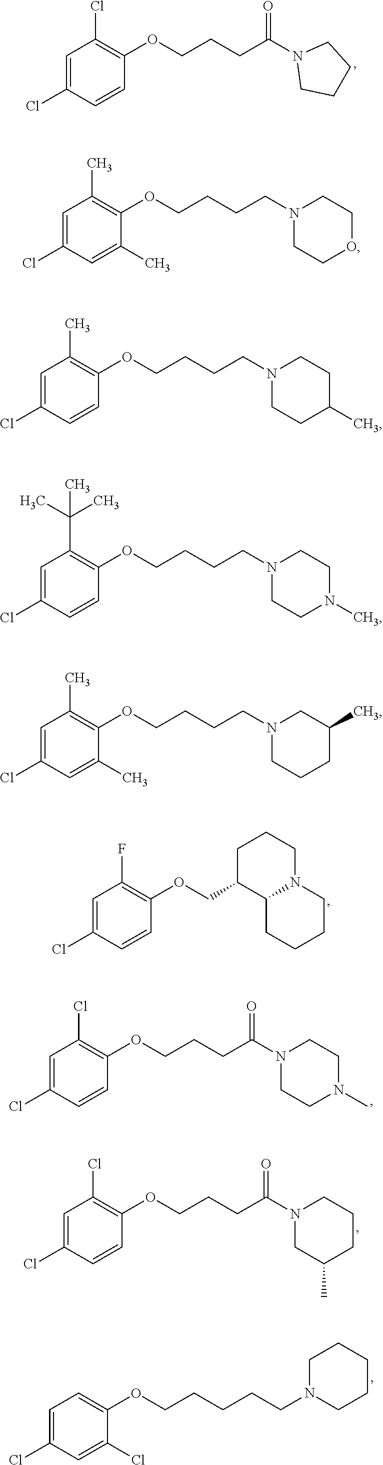

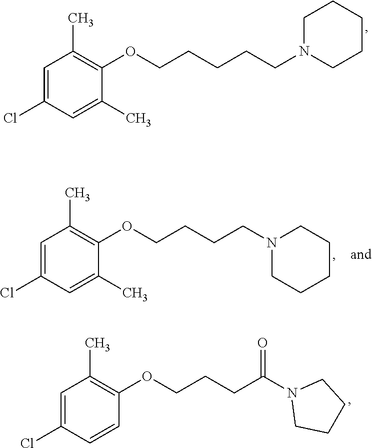 [SMILES: Cc1cc(Cl)cc(C)c1OCCCCCN1CCCCC1 ; Cc1cc(Cl)cc(C)c1OCCCCN1CCCCC1 ; Cc1cc(Cl)ccc1OCCCC(=O)N1CCCC1]

and pharmaceutically acceptable salts thereof.

The present invention further relates to a method of promoting oligodendrocyte precursor cell differentiation, comprising administering to one or more oligodendrocyte precursor cells an effective amount of a compound of formula (III):

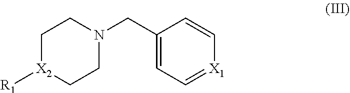

or a pharmaceutically acceptable salt thereof, wherein X₁ and X₂ are each independently selected from CH and N, and R₁ is selected from the group consisting of cycloalkyl and heterocyclyl, each of which is optionally substituted with 1 or 2 substituents.

In some embodiments, the compound of formula (III) is a compound of formula (IIIa):

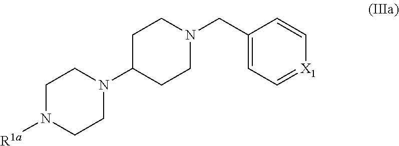

or a pharmaceutically acceptable salt thereof, wherein X₁ is CH or N, and R^{1a} is selected from acyl, arylalkyl, and cycloalkyl, each of which is optionally substituted. In some embodiments, R^{1a} is selected from benzoyl, benzyl, cycloalkyl, and diphenylacetyl, each of which is unsubstituted or substituted with one substituent selected from halo (e.g., fluoro, chloro, or bromo) and alkyl (e.g., methyl).

Examples of compounds having formula (II) that may be used to promote differentiation of oligodendrocyte precursors include:

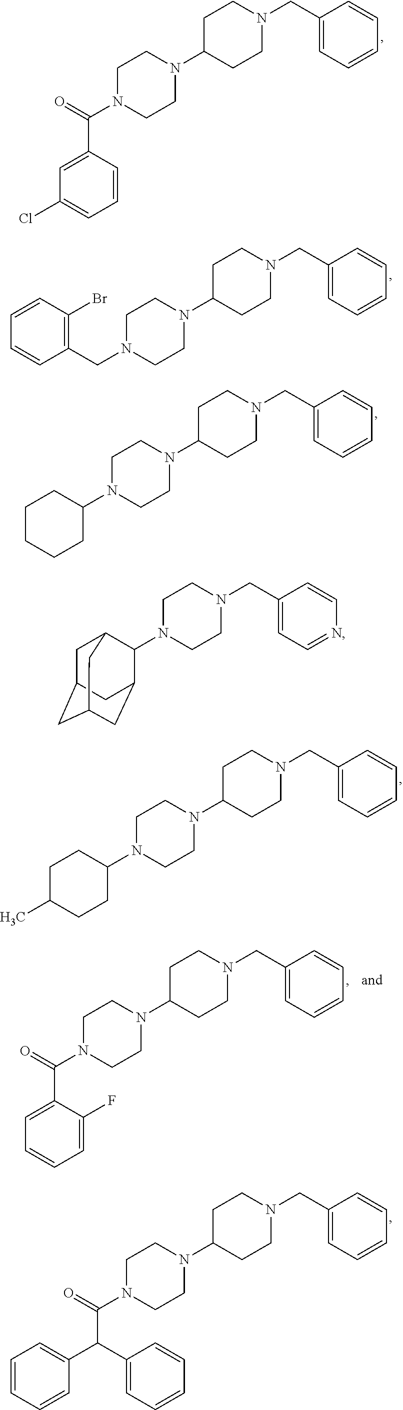

and pharmaceutically acceptable salts thereof.

The present invention further relates to a method of promoting oligodendrocyte precursor cell differentiation, comprising administering to one or more oligodendrocyte precursor cells an effective amount of a compound selected from:

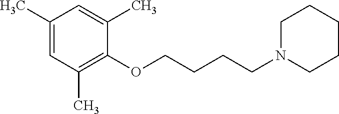 [SMILES: Cc1cc(C)c(OCCCCN2CCCCC2)c(C)c1]

and pharmaceutically acceptable salts thereof.

The present invention further relates to a method of promoting oligodendrocyte precursor cell differentiation, comprising administering to one or more oligodendrocyte precursor cells an effective amount of a compound selected from:

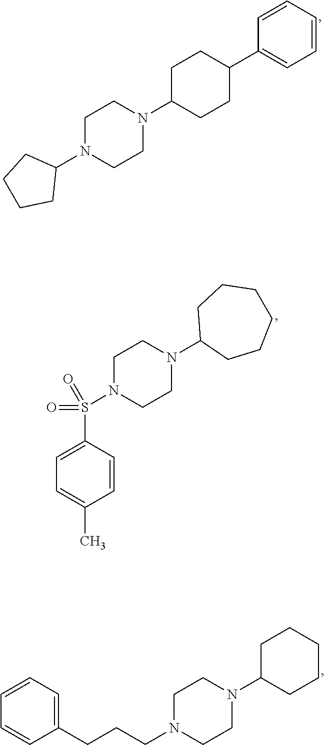 [SMILES: c1ccc(C2CCC(N3CCN(C4CCCC4)CC3)CC2)cc1 ; Cc1ccc(S(=O)(=O)N2CCN(C3CCCCCC3)CC2)cc1 ; c1ccc(CCCN2CCN(C3CCCCC3)CC2)cc1]

and pharmaceutically acceptable salts thereof.

The present invention further relates to a method of promoting oligodendrocyte precursor cell differentiation, comprising administering to one or more oligodendrocyte precursor cells an effective amount of a compound selected from:

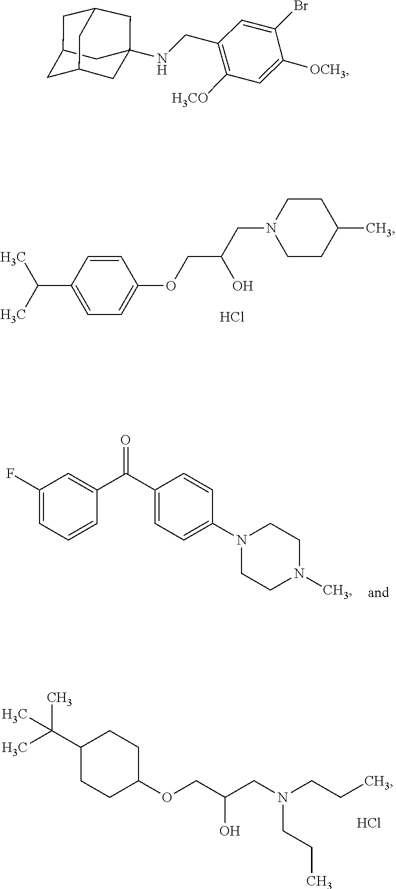 [SMILES: COc1cc(OC)c(CNC23CC4CC(CC(C4)C2)C3)cc1Br ; CC1CCN(CC(O)COc2ccc(C(C)C)cc2)CC1 ; Cl ; CN1CCN(c2ccc(C(=O)c3cccc(F)c3)cc2)CC1 ; CCCN(CCC)CC(O)COC1CCC(C(C)(C)C)CC1 ; Cl]

and pharmaceutically acceptable salts thereof.

The present invention further relates to a method of promoting oligodendrocyte precursor cell differentiation, comprising administering to one or more oligodendrocyte precursor cells an effective amount of a compound selected from:

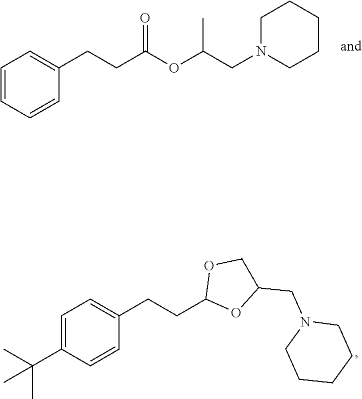 [SMILES: CC(CN1CCCCC1)OC(=O)CCc1ccccc1 ; CC(C)(C)c1ccc(CCC2OCC(CN3CCCCC3)O2)cc1]

and pharmaceutically acceptable salts thereof.

Advantageously, compounds above were found to have high solubility, high hydrophobicity, and produce dramatic up-regulation of the myelin protein PLP/DM20 expression compared to other compounds and controls.

Compounds of the present disclosure may be commercially available or may be synthesized using a variety of methods, which will be evident to those skilled in the art. Compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

When referring to a compound of the invention, applicants intend the term “compound” to encompass not only the specified molecular entities but also their pharmaceutically acceptable, pharmacologically active analogs, including, but not limited to, salts, esters, amides, prodrugs, conjugates, active metabolites, and other such derivatives, analogs, and related compounds.

The oligodendrocyte precursor cell differentiation promoting compounds of the present invention can be provided and administered in the form of pharmaceutical compositions for the in vivo promotion of oligodendrocyte precursor differentiation. The pharmaceutical compositions can be administered to any subject that can experience the beneficial effects of the oligodendrocyte precursor differentiation compounds of the present invention. Foremost among such animals are humans, although the present invention is not intended to be so limited.

Pharmaceutical compositions for use in the methods of the present invention preferably have a therapeutically effective amount of the compound or salts thereof in a dosage in the range of 0.01 to 1,000 mg/kg of body weight of the subject, and more preferably in the range of from about 10 to 100 mg/kg of body weight of the patient.

The overall dosage will be a therapeutically effective amount depending on several factors including the overall health of a subject, the subject's disease state, severity of the condition, the observation of improvements and the formulation and route of administration of the selected agent(s). Determination of a therapeutically effective amount is within the capability of those skilled in the art. The exact formulation, route of administration and dosage can be chosen by the individual physician in view of the subject's condition.

The present invention provides a method of treating diseases in a subject by promoting the differentiation of oligodendrocyte precursors in a subject. The method includes administering to the subject in need thereof a therapeutically effective amount of a pharmaceutical compound in accordance with the present invention. As described above, one or more of the compounds can be administered in association with one or more non-toxic, pharmaceutically acceptable carriers and/or diluents and/or adjuvants and if desired other active ingredients.

The “therapeutically effective amount” of compounds and salts thereof used in the methods of the present invention varies depending upon the manner of administration, the age and body weight of the subject, and the condition of the subject to be treated, and ultimately will be decided by those skilled in the art The term “therapeutically effective amount” refers to an amount (dose) effective in treating a subject, having, for example, a neurodegenerative disease (e.g. multiple sclerosis).

“Treating” or “treatment” as used herein, refers to the reduction in severity and/or frequency of symptoms, elimination of symptoms and/or underlying cause, prevention of the occurrence of symptoms and/or their underlying cause, and improvement or remediation of disease. Such treatment need not necessarily completely ameliorate the disease. For example, treatment of a subject with a neurodegenerative disease by administration of oligodendrocyte precursor differentiation compounds of the present invention can encompass inhibiting or causing regression of the disease. Further, such treatment can be used in conjunction with other traditional treatments for neurodegenerative diseases known to those of skill in the art.

The pharmaceutical compositions of the present invention can be administered to a subject by any means that achieve their intended purpose. For example, administration can be by parenteral, subcutaneous, intravenous, intraarticular, intrathecal, intramuscular, intraperitoneal, or intradermal injections, or by transdermal, buccal, oromucosal, ocular routes or via inhalation. Alternatively, or concurrently, administration can be by the oral route.

Formulation of the pharmaceutical compounds for use in the modes of administration noted above (and others) are known in the art and are described, for example, in Remington's Pharmaceutical Sciences (18th edition), cd. A. Gennaro, 1990, Mack Publishing Company, Easton, Pa. (also see, e.g., M. J. Rathbone, cd. Oral Mucosal Drug Delivery, Drugs and the Pharmaceutical Sciences Scries, Marcel Dekker, Inc., N.Y., U.S.A., 1996; M. J. Rathbone et al., Modified-Release Drug Delivery Technology, Drugs and the Pharmaceutical Sciences Series, Marcel Dekker, Inc., N.Y., U.S.A., 2003; Ghosh et a)., cds., Drug Delivery to the Oral Cavity, Drugs and the Pharmaceutical Sciences Series, Marcel Dekker, Inc., N.Y., U.S.A., 2005; and Mathiowitz et al., eds., Bioadhesive Drug Delivery Systems, Drugs and the Pharmaceutical Sciences Series, Marcel Dekker, Inc., N.Y., U.S.A., 1999. Compounds of the invention can be formulated into pharmaceutical compositions containing pharmaceutically acceptable non-toxic excipients and carriers. The excipients are all components present in the pharmaceutical formulation other than the active ingredient or ingredients. Suitable excipients and carriers useful in the present invention are composed of materials that are considered safe and effective and may be administered to an individual without causing undesirable biological side effects, or unwanted interactions with other medications. Suitable excipients and carriers are those, which are composed of materials that will not affect the bioavailability and performance of the agent. As generally used herein “excipient” includes, but is not limited to surfactants, emulsifiers, emulsion stabilizers, emollients, buffers, solvents, dyes, flavors, binders, fillers, lubricants, and preservatives. Suitable excipients include those generally known in the art such as the “Handbook of Pharmaceutical Excipients” 4th Ed., Pharmaceutical Press, 2003.

The compounds in accordance with the present invention can be administered to a subject to treat neurodegenerative conditions. A neurodegenerative disease, as contemplated for treatment by methods of the present invention, can arise from but is not limited to stroke, heat stress, head and spinal cord trauma (blunt or infectious pathology), and bleeding that occurs m the brain. Examples of neurodegenerative disorders contemplated include Alexander disease, Alper's disease, Amyotrophic lateral sclerosis, Ataxia telangiectasia, Spielmeyer-Vogt-Sjogren-Batten disease, Bovine spongiform encephalopathy, Canavan disease, Cockayne syndrome, Corticobasal degeneration, Creutzfeldt-Jakob disease, Huntington's Disease, HIV-associated dementia, Kennedy's disease, Krabbe disease, Lewy body dementia, Spinocerebellar ataxias, Multiple Sclerosis, Multiple system atrophy, Neuroborreliosis, Parkinson's disease, Pelizaeus-Merzbacher disease, Pick's disease, Primary lateral sclerosis, Prion diseases, Refsunus disease, Sandhoff disease, Schilder's disease, Spinal muscular atrophy, Steele-Richardson-Olszewski disease, and tabes dorsalis.

The neurodegenerative disease contemplated for treatment by some aspects of the present invention can include a myelin related disorder. Myelin disorders can include any disease, condition (e.g., those occurring from traumatic spinal cord injury and cerebral infarction), or disorder related to demyelination, remyelination, or dysmyelination in a subject. A myelin related disorder as used herein can arise from a myelination related disorder or demyelination resulting from a variety of neurotoxic insults. “‘Demyelination” as used herein, refers to the act of demyelinating, or the loss of the myelin sheath insulating the nerves, and is the hallmark of some neurodegenerative autoimmune diseases, including multiple sclerosis, transverse myelitis, chronic inflammatory demyelinating polyneuropathy, and Guillain-Barre Syndrome. Leukodystrophies are caused by inherited enzyme deficiencies, which cause abnormal formation, destruction, and/or abnormal turnover of myelin sheaths within the CNS white matter. Both acquired and inherited myelin disorders share a poor prognosis leading to major disability. Thus, some embodiments of the present invention can include methods for the treatment of neurodegenerative autoimmune diseases in a subject. The term “remyelination,” as used herein, refers to the re-generation of the nerve's myelin sheath by replacing myelin producing cells or restoring their function.

One particular aspect of the present invention contemplates the treatment of multiple sclerosis in a subject. The method includes administering to the subject a therapeutically effective amount of one or more oligodendrocyte differentiation promoting compound(s) described above.

Multiple sclerosis (MS) is the most common demyelinating disease. In multiple sclerosis, the body's failure to repair myelin is thought to lead to nerve damage, causing multiple sclerosis associated symptoms and increasing disability. It is contemplated that methods of the present invention can promote oligodendrocyte precursor cell differentiation in a subject, therefore leading to endogenous remyelination.

Another strategy for treating a subject suffering from a neurodegenerative disease or disorder is to administer a therapeutically effective amount of a compound described herein along with a therapeutically effective amount of additional oligodendrocyte differentiation inducing agent(s) and/or anti-neurodegenerative disease agent. Examples of anti-neurodegenerative disease agents include L-dopa, cholinesterase inhibitors, anticholinergics, dopamine agonists, steroids, and immunomodulators including interferons, monoclonal antibodies, and glatiramer acetate.

Therefore, in a further aspect of the invention, the oligodendrocyte precursor differentiation inducing agents can be administered as part of a combination therapy with adjunctive therapies for treating neurodegenerative and myelin related disorders.

The phrase “combination therapy” embraces the administration of the oligodendrocyte precursor differentiation inducing agents and a therapeutic agent as part of a specific treatment regimen intended to provide a beneficial effect from the co-action of these therapeutic agents. When administered as a combination, the oligodendrocyte precursor differentiation inducing agents and a therapeutic agent can be formulated as separate compositions. Administration of these therapeutic agents in combination typically is carried out over a defined time period (usually minutes, hours, days or weeks depending upon the combination selected).

“Combination therapy” is intended to embrace administration of these therapeutic agents in a sequential manner, that is, wherein each therapeutic agent is administered at a different lime, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different therapeutic agent) and non-drug therapies (e.g., surgery).

In another aspect of the invention, the therapeutic agents administered in a combination therapy with the oligodendrocyte differentiation inducing agents can include at least one anti-neurodegenerative agent selected from the group consisting of an immunotherapeutic agent.

An immunotherapeutic agent for use in the methods of the present invention can include therapies which target the immune component of the disease and/or the acute inflammatory response evidenced during an acute attack in remitting-relapsing multiple sclerosis. Examples include, but are not limited to immunomodulators such as interferon beta-1a and beta-1b (Avonex and Betaseron respectively), nataliumab (Copaxone) natalizumab (Tysabri), glatiramer acetate (Copaxone) or mitoxantrone.

It should be understood that the methods described herein may be carried out in a number of ways and with various modifications and permutations thereof that are well known in the art. It may also be appreciated that any theories set forth as to modes of action should not be construed as limiting this invention in any manner, but are presented such that the methods of the invention can be more fully understood.

### EXAMPLES

### Example 1

### Large-Scale Cell-Based Phenotypic Screening and Clustering of Hit Series

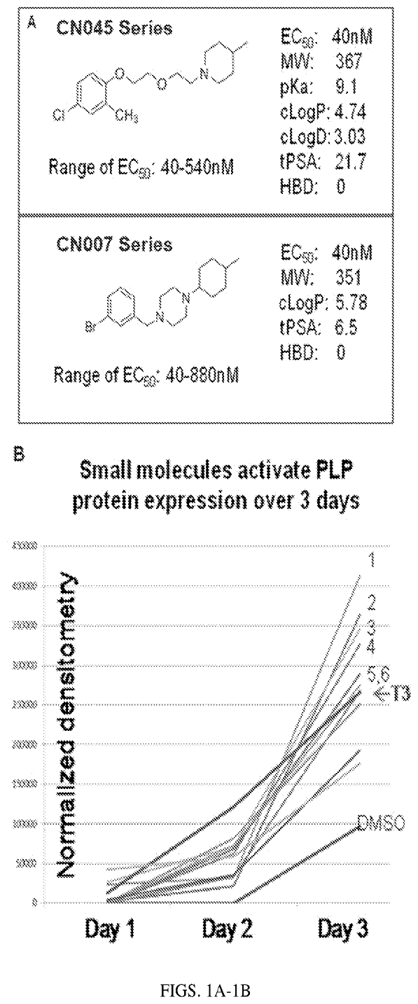

Using the 96-well OPC-screening platform, a CNS-bias library of 20,000 drug-like small molecule compounds (Cambridge Co., San Diego, Calif.) was screened, using DMSO as a negative control and T3 (triiodothyronine) as a positive control. Initial screening was at 10 μM, and 390 primary hits were identified. Further testing confirmed that 57 compounds produced at least 50% response of T3. Among those, 43 small molecules had an EC₅₀ lower than 1 μM and can be clustered into at least 6 chemical series. In particular, CN045 and CN007 series contain many analogs with EC₅₀ below 0.1 μM (FIG. 1A).

### Example 2

### Compounds Activated Multiple Oligodendrocyte Differentiation Markers

OPCs were treated with top “hit” compounds from different chemical groups and cell lysates were extracted for Western blotting analysis using antibodies to PLP, CNPase and housekeeping protein GAPDH (as a loading control) at day 4. Compared with DMSO-only control, treatment with “hit” compounds showed higher levels of PLP, DM20 and CNPase. To further determine the dynamics of PLP expression, lysates were collected following 1-3 days of treatment, and the level of PLP protein was determined using quantitative Western blotting analysis. Treatment with different compounds (different lines in FIG. 1B) induced much higher levels of PLP protein expression than DMSO and with different slopes.

### Example 3

### Selection of CN045 as a Lead Compound

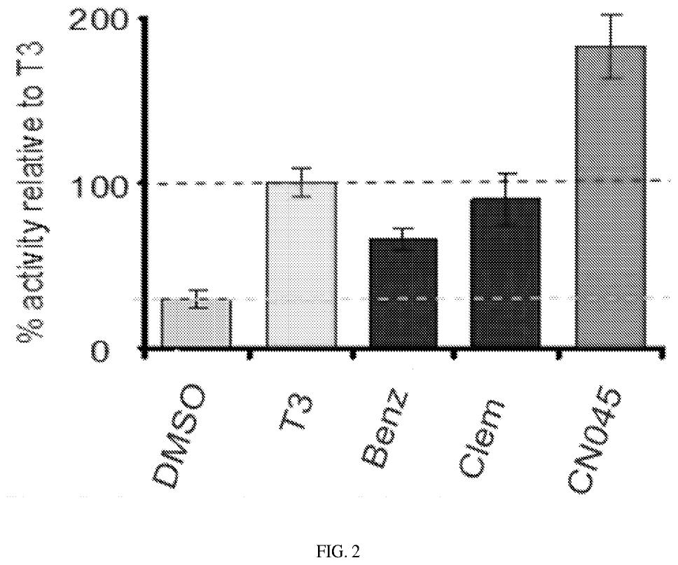

Among the positive hit series, CN045 stands out because of the following features: high efficacy and potency (see below), low toxicity (>80% viability up to 30 μM in MTT assay), good solubility (>500 μM), and promising physiochemical properties. In addition, when compared with two anti-muscarinic drugs (benztropine and clemastine) coming out of the recent repurposed screens (Deshmukh et al. Nature 2013, 502(7471), 327-332; Mei et al. Nat. Med. 2014, 20, 954-960) in the same OPC assay, CN045 showed almost twice the efficacy of both of these drugs or T3 when tested at multiple concentrations (FIG. 2). Based on these results, CN045 was selected as a lead candidate. The second best compound, CN007 (in FIG. 1A), which also shows an EC₅₀ of 40 nM, was chosen as a backup compound series.

### Example 4

### CN045 Activates Cellular Differentiation Program in Oligodendrocyte

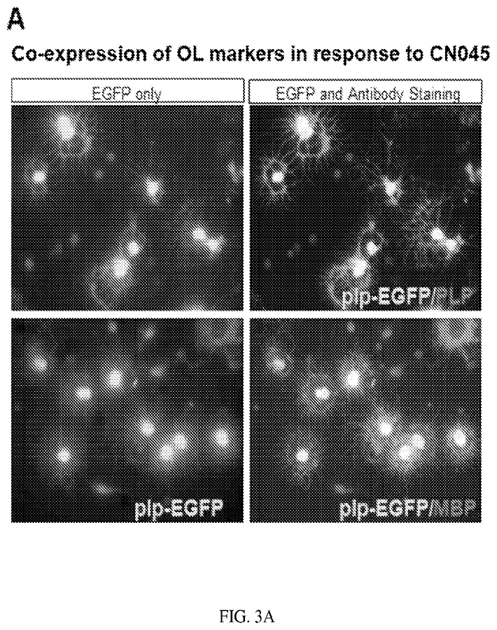

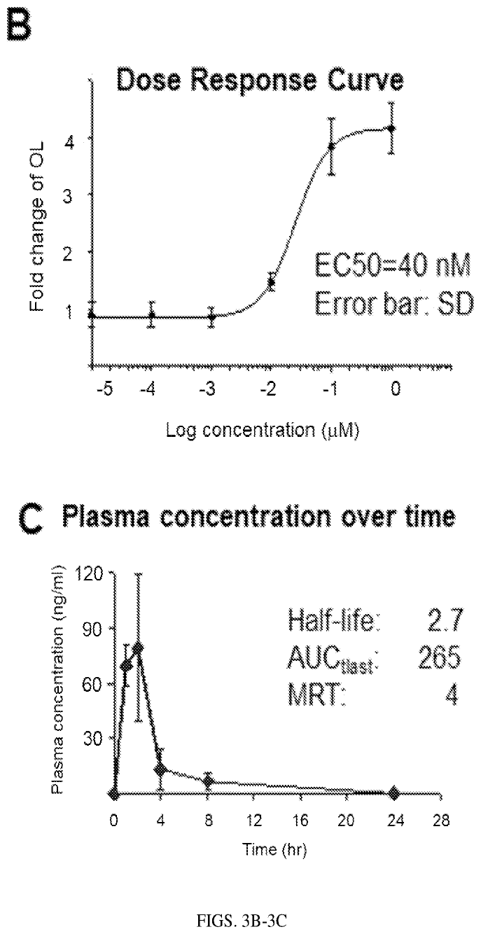

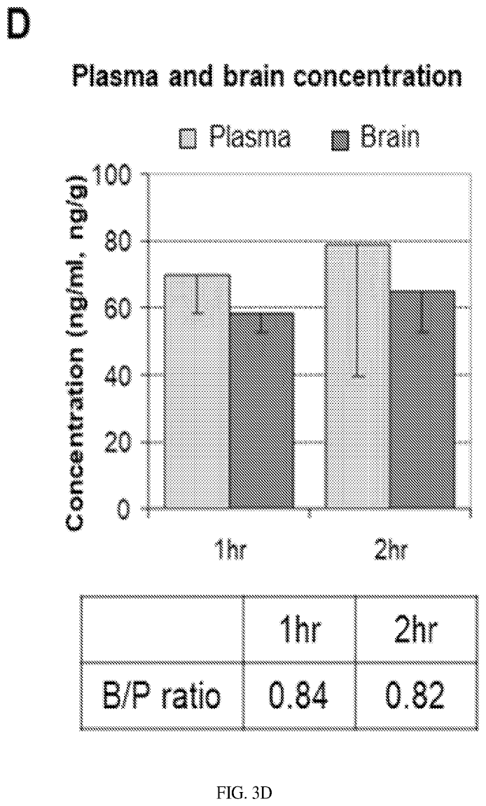

To ascertain that CN045 activated multiple oligodendrocyte markers in the same cells, OPCs were treated with CN045 for 4 days and immunostained cells with PLP and MBP (FIG. 3A). All EGFP+ cells were co-labeled by PLP and MBP antibodies. The observation that EGFP-positive cells displayed typical lily-pad morphology further confirm that the “hit” compounds activate oligodendrocyte differentiation at both molecular and morphological levels. Finally, similar activation of oligodendrocyte markers was observed when wild-type OPCs were treated with the “hit” compounds, eliminating the possibility that expression of the EGFP construct affected the results.

### Example 5

### Preliminary Pharmacokinetic Analysis of CN045

CN045 was delivered via i.p. injections to 5 groups of C57BU6J mice (n=3 per group) at a dose of 10 mg/kg body weight, and plasma and brain tissues were collected at 1, 2, 4, 8 and 24 hr post-injection. The concentration of CN045 was determined through tandem mass spectrometry (LC/MS/MS). Peak concentration was found ˜2 hr following injection. When the results were plotted and modeled using PK package of the R software, CN045 was found to have a non-compartmental half-life of 2.7 hr (FIG. 3C). To further determine whether CN045 could penetrate the blood-brain barrier, the concentration of CN045 was determined in brain homogenate vs. plasma (FIG. 3D). The brain/plasma (8/P) ratio reached 0.8, suggesting that CN045 enters the brain.

### Example 6

### CN045 Significantly Increased Remyelination in the Cortex, Hippocampus and Corpus Callosum in the Cuprizone Mouse Model of Demyelination and Remyelination

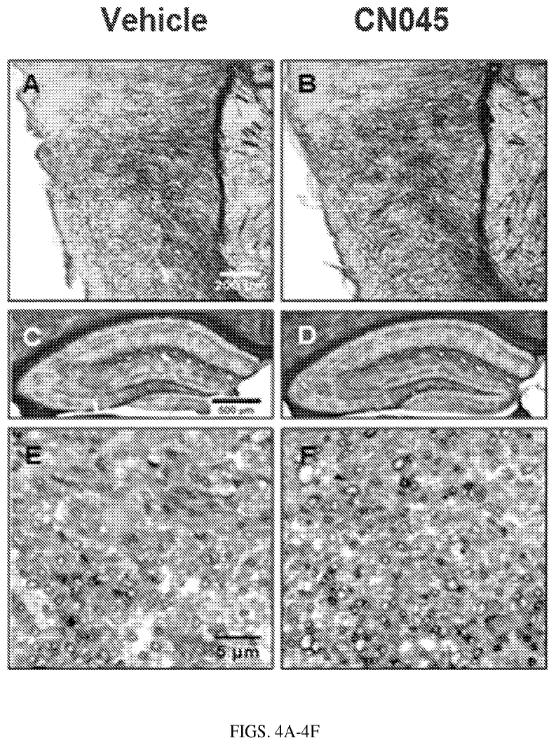

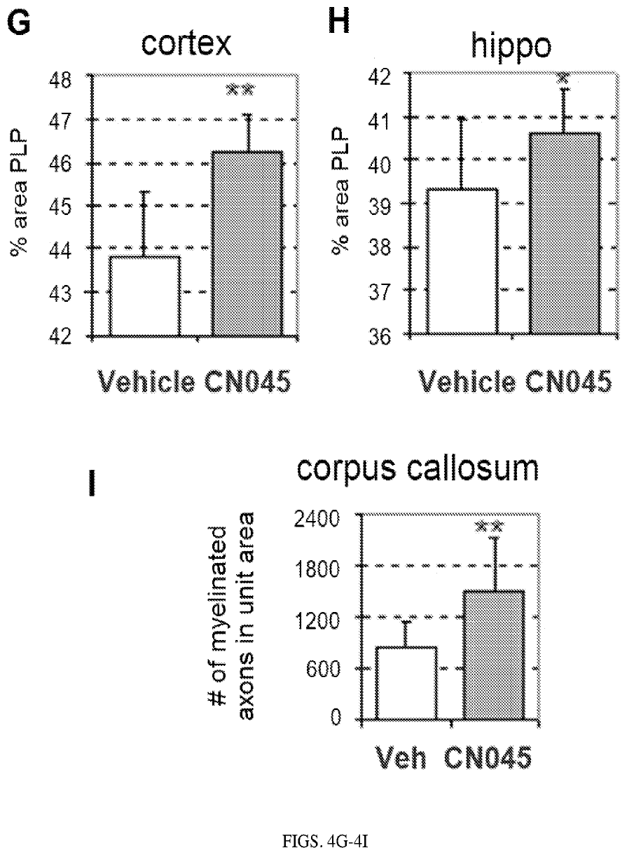

To assess the ability of CN045 to increase remyelination without impacting the immune system, CN045 was delivered to mice for 6 weeks (n=12 per group) that were demyelinated using a cuprizone/rapamycin demyelination protocol, described in more detail in Bai et al. Exp. Neurol. 2016; 283(Pt A): 330-340, which is incorporated herein by reference in its entirety. The compound CN045 was i.p. injected daily for 6 weeks at 10 mg/kg body weight. At the end of the treatment period, mice were perfused and different tissues analyzed for remyelination. In cortex and hippocampus, PLP antibody staining was used to assess remyelination. In corpus callosum, the number of myelinated axons in semi-thin (1 μm) Epon sections was quantified. Compared to the control, CN045 significantly increased remyelination in cortex (p<0.01, Student's t-test) (FIGS. 4A, 4B, 4G), hippocampus (p<0.05) (FIGS. 4C, 4D, 4H) and corpus callosum (p<0.01) (FIGS. 4E, 4F, 4I). This in vivo animal data further validates a strategy of using cell-based phenotypic screening to identify remyelination compounds.

### Example 7

### Compounds and Data

Specific compounds and associated data are shown below in Tables 1 and 2.

TABLE 1
CN045, CN007 and Analogs

| Cmpd | | | | | | | | | CNS |
|---|---|---|---|---|---|---|---|---|---|
| Num- | | EC₅₀ | | | | | | | MPO |
| ber | Structures | μM | MW | pKa | cLogP | cLogD | tPSA | HBD | Score |
| CN045 | 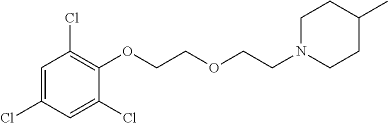 [SMILES: CC1CCN(CCOCCOc2c(Cl)cc(Cl)cc2Cl)CC1] | 0.040 | 367 | 9.10 | 4.74 | 3.03 | 21.7 | 0 | 3.1 |
| CN049 | 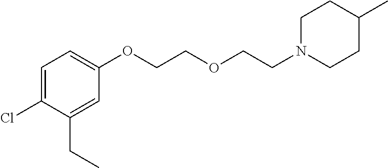 [SMILES: CCc1cc(OCCOCCN2CCC(C)CC2)ccc1Cl] | 0.165 | 326 | 9.10 | 4.49 | 2.78 | 21.7 | 0c | 3.4 |
| CN918 | 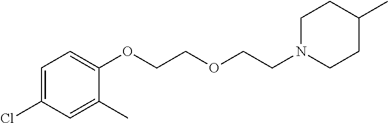 [SMILES: Cc1cc(Cl)ccc1OCCOCCN1CCC(C)CC1] | 0.193 | 312 | 9.10 | 4.04 | 2.34 | 21.7 | 0 | 3.8 |
| CN570 | 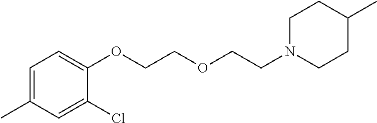 | 0.200 | 312 | 9.10 | 4.04 | 2.34 | 21.7 | 0 | 3.8 |
| CN265 | 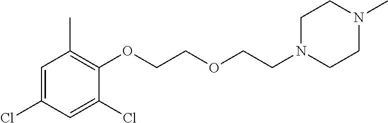 [SMILES: Cc1cc(Cl)cc(Cl)c1OCCOCCN1CCN(C)CC1] | 0.540 | 347 | 8.29 | 3.36 | 2.42 | 24.9 | 0 | 4.7 |
| CN007 | 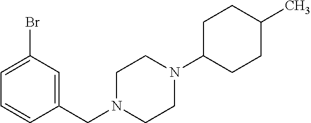 [SMILES: CC1CCC(N2CCN(Cc3cccc(Br)c3)CC2)CC1] | 0.040 | 351 | — | 5.78 | — | 6.5 | 0 | — |

TABLE 2
Additional Compounds and Data

| Cmpd | | EC₅₀ | | | | |
|---|---|---|---|---|---|---|
| Number | Structures | μM | MW | cLogP | tPSA | HBD |
| CN317 | 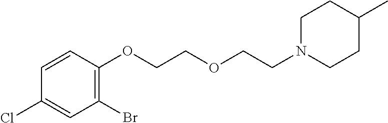 [SMILES: CC1CCN(CCOCCOc2ccc(Cl)cc2Br)CC1] | — | 377 | 3.93 | 21.7 | 0 |
| CN773 | 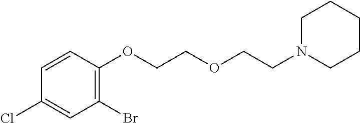 [SMILES: Clc1ccc(OCCOCCN2CCCCC2)c(Br)c1] | — | 363 | 3.66 | 21.7 | 0 |
| CN028 | 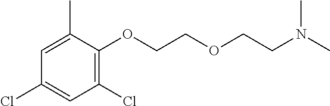 [SMILES: Cc1cc(Cl)cc(Cl)c1OCCOCCN(C)C] | — | 292 | 2.75 | 21.7 | 0 |
| CN857 | 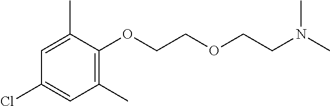 [SMILES: Cc1cc(Cl)cc(C)c1OCCOCCN(C)C] | — | 272 | 2.49 | 21.7 | 0 |
| CN774 | 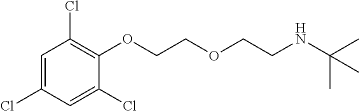 [SMILES: CC(C)(C)NCCOCCOc1c(Cl)cc(Cl)cc1Cl] | — | 341 | 3.89 | 30.49 | 1 |
| CN409 | 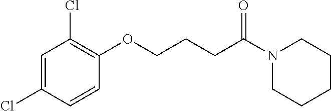 [SMILES: O=C(CCCOc1ccc(Cl)cc1Cl)N1CCCCC1] | — | 316 | 4.19 | 29.54 | 0 |
| CN276 | 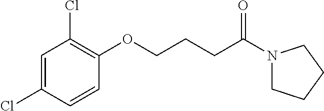 [SMILES: O=C(CCCOc1ccc(Cl)cc1Cl)N1CCCC1] | — | 302 | 3.85 | 29.54 | 0 |
| CN644 | 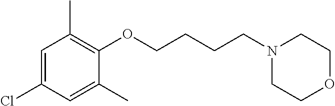 [SMILES: Cc1cc(Cl)cc(C)c1OCCCCN1CCOCC1] | 0.27 | 298 | 3.46 | 21.7 | 0 |
| CN435 | 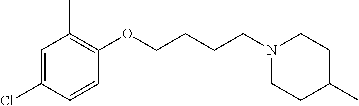 [SMILES: Cc1cc(Cl)ccc1OCCCCN1CCC(C)CC1] | — | 296 | 4.55 | 12.47 | 0 |
| CN950 | 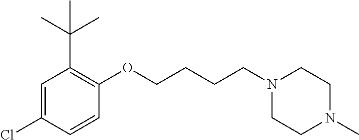 [SMILES: CN1CCN(CCCCOc2ccc(Cl)cc2C(C)(C)C)CC1] | — | 339 | 4.46 | 15.71 | 0 |
| CN157 | 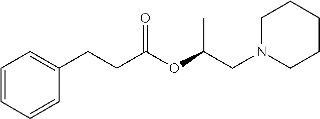 [SMILES: C[C@@H](CN1CCCCC1)OC(=O)CCc1ccccc1] | — | 275 | 3.22 | 29.54 | 0 |
| CN329 | 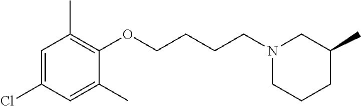 [SMILES: Cc1cc(Cl)cc(C)c1OCCCCN1CCC[C@H](C)C1] | 0.042 | 310 | 4.89 | 12.47 | 0 |
| CN566 | 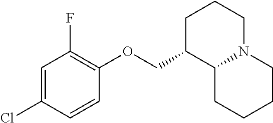 [SMILES: Fc1cc(Cl)ccc1OC[C@@H]1CCCN2CCCC[C@H]12] | 0.34 | 298 | 3.86 | 12.47 | 0 |
| CN838 | 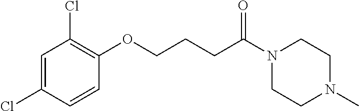 [SMILES: CN1CCN(C(=O)CCCOc2ccc(Cl)cc2Cl)CC1] | — | 331 | 3.14 | 32.78 | 0 |
| CN855 | 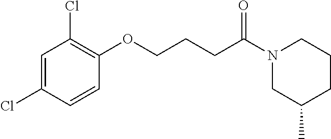 [SMILES: C[C@H]1CCCN(C(=O)CCCOc2ccc(Cl)cc2Cl)C1] | — | 330 | 4.46 | 29.54 | 0 |
| CN526 | 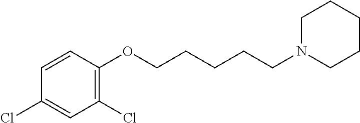 [SMILES: Clc1ccc(OCCCCCN2CCCCC2)c(Cl)c1] | — | 316 | 5.00 | 12.47 | 0 |
| CN956 | 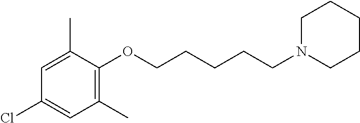 [SMILES: Cc1cc(Cl)cc(C)c1OCCCCCN1CCCCC1] | — | 310 | 5.08 | 12.47 | 0 |
| CN478 | 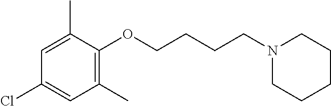 [SMILES: Cc1cc(Cl)cc(C)c1OCCCCN1CCCCC1] | 0.078 | 296 | 4.63 | 12.47 | 0 |
| CN936 | 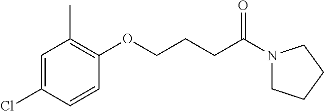 [SMILES: Cc1cc(Cl)ccc1OCCCC(=O)N1CCCC1] | — | 282 | 3.59 | 29.54 | 0 |
| CN839 | 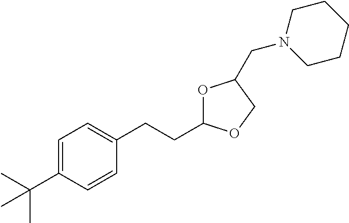 [SMILES: CC(C)(C)c1ccc(CCC2OCC(CN3CCCCC3)O2)cc1] | — | 332 | 4.44 | 21.7 | 0 |

The contents of all references, patent applications, patents, and published patent applications cited throughout this application are hereby incorporated by reference in their entirety.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

1. A method of promoting oligodendrocyte precursor cell differentiation, comprising administering to one or more oligodendrocyte precursor cells an effective amount of a compound of formula (IV): 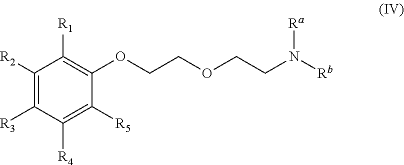
or a pharmaceutically acceptable salt thereof, wherein: X is selected from N and CH; R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R^{a} and R^{b} are each independently selected from hydrogen and alkyl, or R^{a} and R^{b} are taken together with the nitrogen atom to which they are attached to form an optionally substituted heterocyclyl.

2. The method of claim 1, wherein R₁, R₂, R₃, R₄, and R₅ are each independently selected from the group consisting of hydrogen, alkyl, and halo.

3. The method of claim 1, wherein the compound of formula (IV) is a compound selected from the group consisting of: 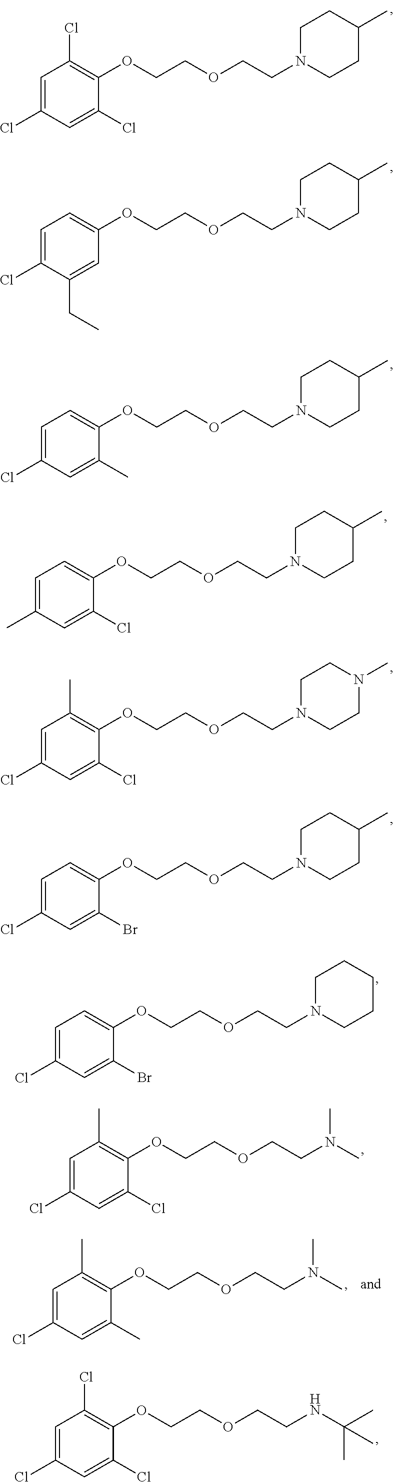
and pharmaceutically acceptable salts thereof.

4. A method of promoting oligodendrocyte precursor cell differentiation, comprising administering to one or more oligodendrocyte precursor cells an effective amount of a compound of formula (I): 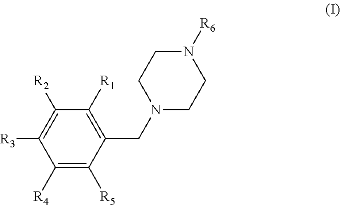
or a pharmaceutically acceptable salt thereof, wherein: R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R⁶ is a group of formula —(CH₂)ₙ-A, wherein n is 0 or 1, and A is selected from aryl, heteroaryl, cycloalkyl, cycloalkenyl, and heterocyclyl, wherein the aryl, heteroaryl, cycloalkyl, cycloalkenyl, and heterocyclyl are optionally substituted with 1, 2, or 3 substituents.

5. The method of claim 4, wherein the compound of formula (I) is a compound selected from the group consisting of: 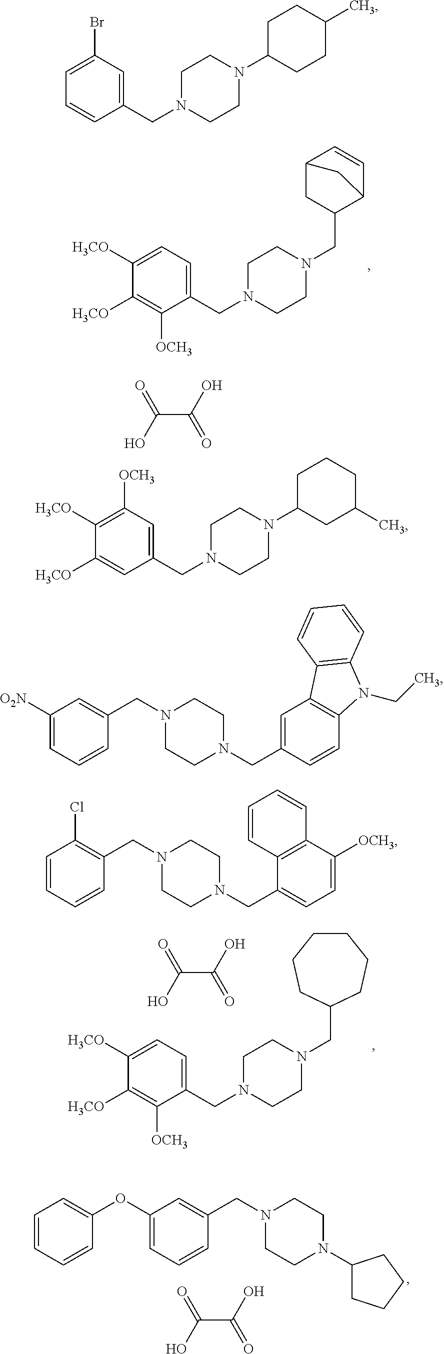 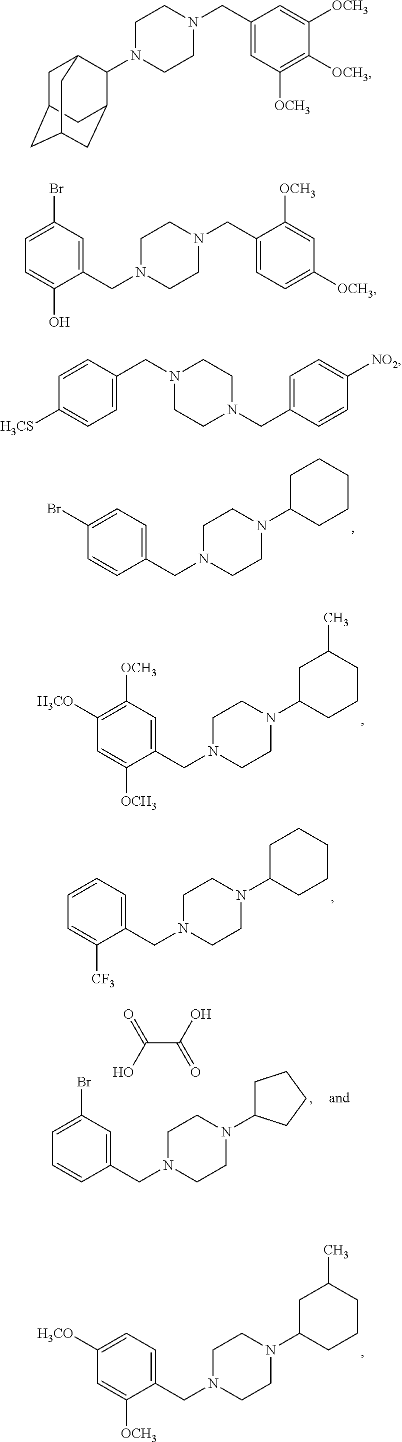
and pharmaceutically acceptable salts thereof.

6. A method of promoting oligodendrocyte precursor cell differentiation, comprising administering to one or more oligodendrocyte precursor cells an effective amount of a compound of formula (II): 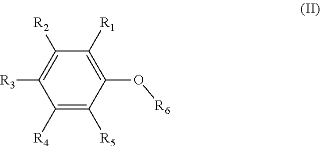
or a pharmaceutically acceptable salt thereof, wherein: R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; wherein R₁ and R₂, R₂ and R₃, R₃ and R₄, or R₄ and R₅ are optionally taken together with the carbon atoms to which they are attached to form a ring; and R₆ is a group of formula —(CH₂)ₙ—(C(O))ₘ—NR^{a}R^{b}, wherein: m is 0 or 1; n is 1, 2, 3, 4, 5, or 6; and R^{a} and R^{b} are each independently selected from alkyl, or R^{a} and R^{b} are taken together with the nitrogen atom to which they are attached to form an optionally substituted heterocyclyl.

7. The method of claim 6, wherein the compound of formula (II) is a compound selected from the group consisting of: 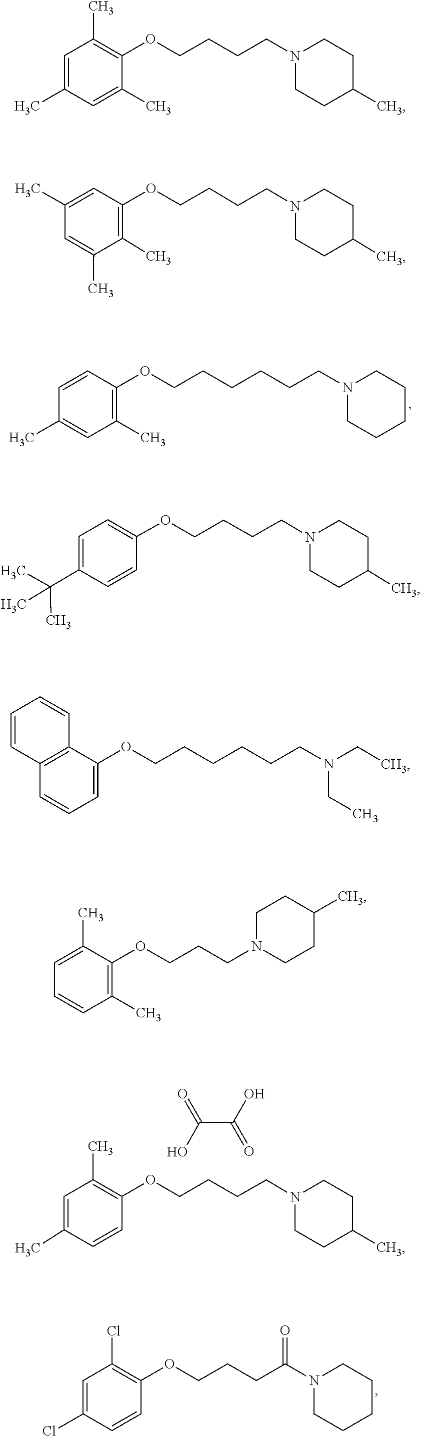 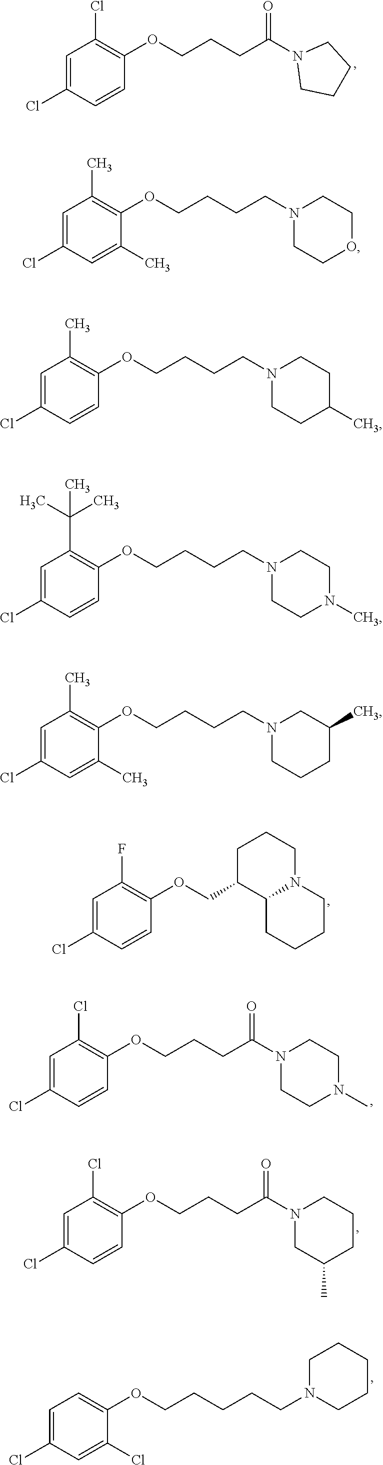 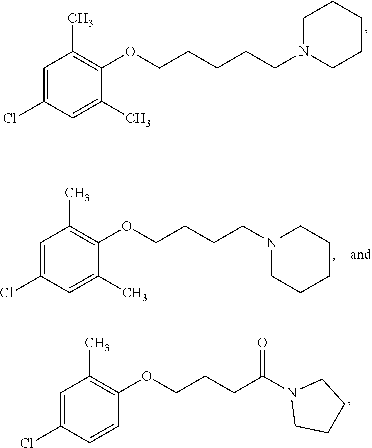 [SMILES: Cc1cc(Cl)cc(C)c1OCCCCCN1CCCCC1 ; Cc1cc(Cl)cc(C)c1OCCCCN1CCCCC1 ; Cc1cc(Cl)ccc1OCCCC(=O)N1CCCC1]
and pharmaceutically acceptable salts thereof.

8. A method of promoting oligodendrocyte precursor cell differentiation, comprising administering to one or more oligodendrocyte precursor cells an effective amount of a compound of formula (III): 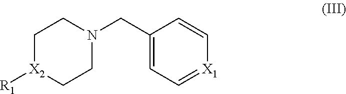
or a pharmaceutically acceptable salt thereof, wherein X₁ and X₂ are each independently selected from CH and N, and R₁ is selected from the group consisting of cycloalkyl and heterocyclyl, each of which is optionally substituted with 1 or 2 substituents.

9. The method of claim 8, wherein the compound of formula (III) is a compound selected from the group consisting of:

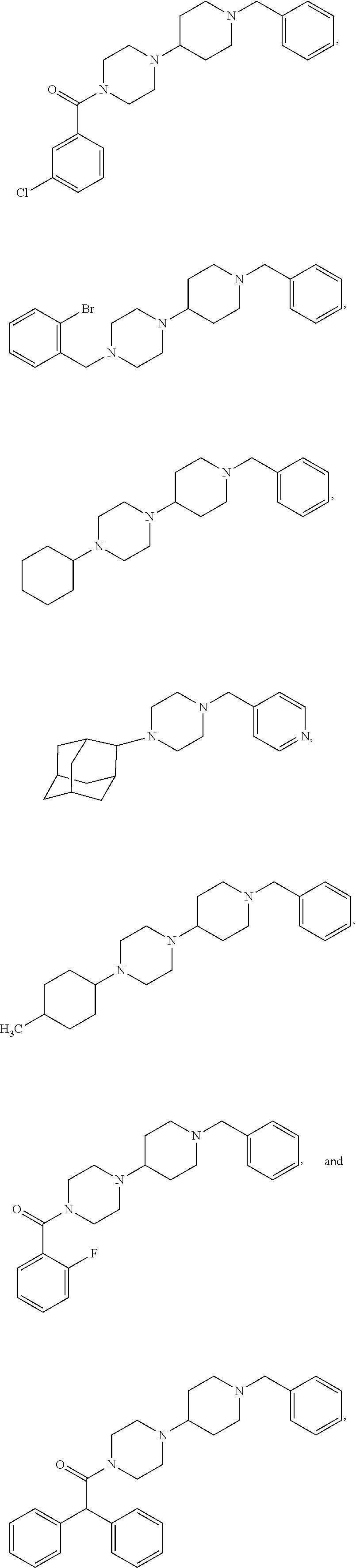

10. A method of treating a neurodegenerative disease in a subject in need thereof, comprising administering to the subject a therapeutically effective amount of a compound of formula (IV): 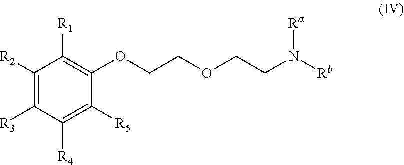
or a pharmaceutically acceptable salt thereof, wherein: X is selected from N and CH; R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R^{a} and R^{b} are each independently selected from hydrogen and alkyl, or R^{a} and R^{b} are taken together with the nitrogen atom to which they are attached to form an optionally substituted heterocyclyl.

11. The method of claim 10, wherein R₁, R₂, R₃, R₄, and R₅ are each independently selected from the group consisting of hydrogen, alkyl, and halo.

12. The method of claim 10, wherein the compound of formula (IV) is a compound selected from the group consisting of: 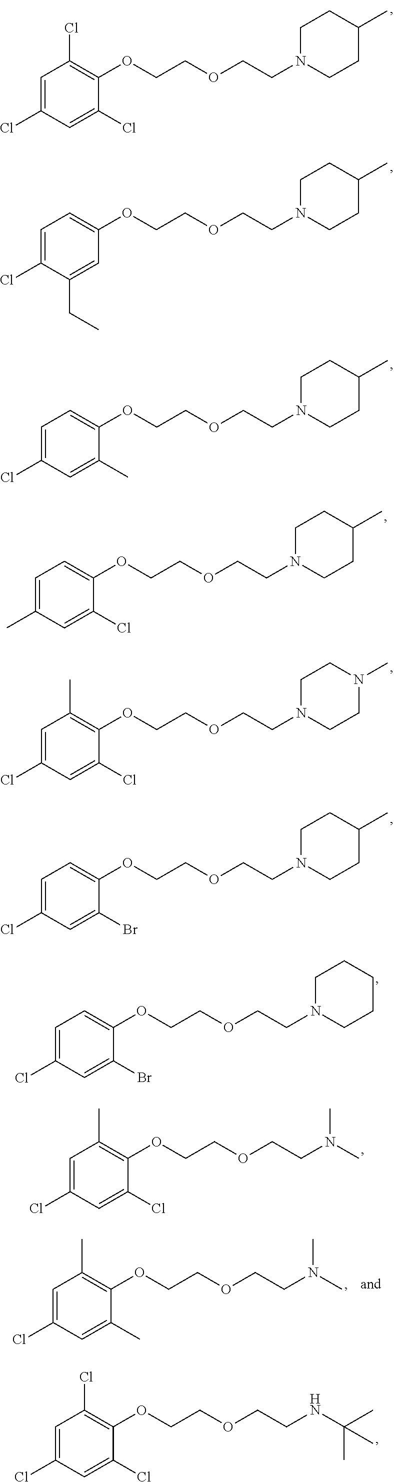
and pharmaceutically acceptable salts thereof.

13. A method of treating a neurodegenerative disease in a subject in need thereof, comprising administering to the subject a therapeutically effective amount of a compound of formula (I): 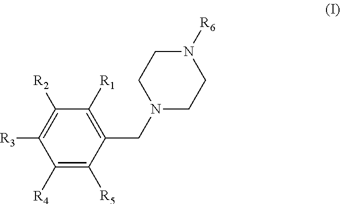
or a pharmaceutically acceptable salt thereof, wherein R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; and R⁶ is a group of formula —(CH₂)ₙ-A, wherein n is 0 or 1, and A is selected from aryl, heteroaryl, cycloalkyl, cycloalkenyl, and heterocyclyl, wherein the aryl, heteroaryl, cycloalkyl, cycloalkenyl, and heterocyclyl are optionally substituted with 1, 2, or 3 substituents.

14. The method of claim 13, wherein the compound of formula (I) is a compound selected from the group consisting of: 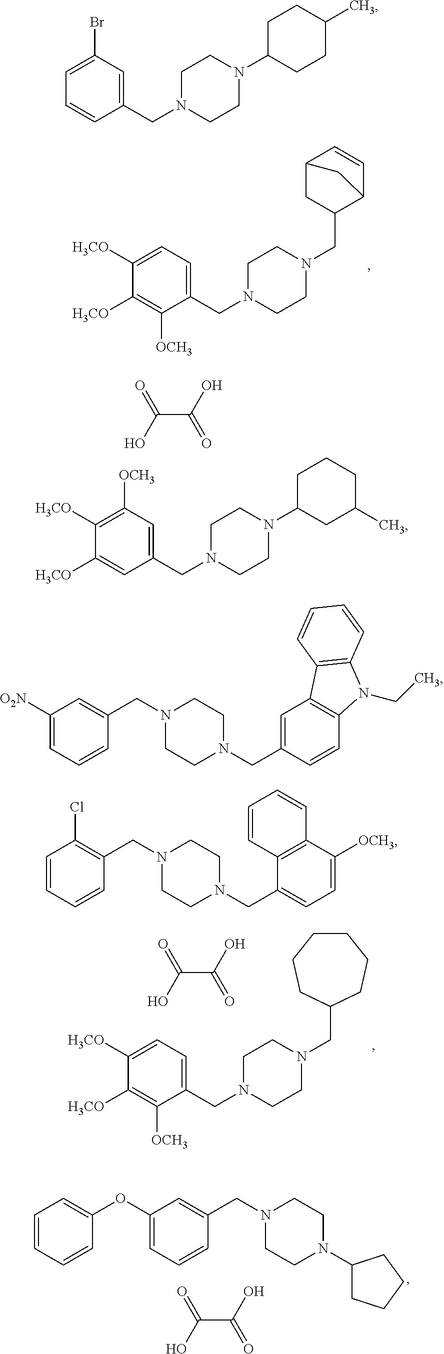 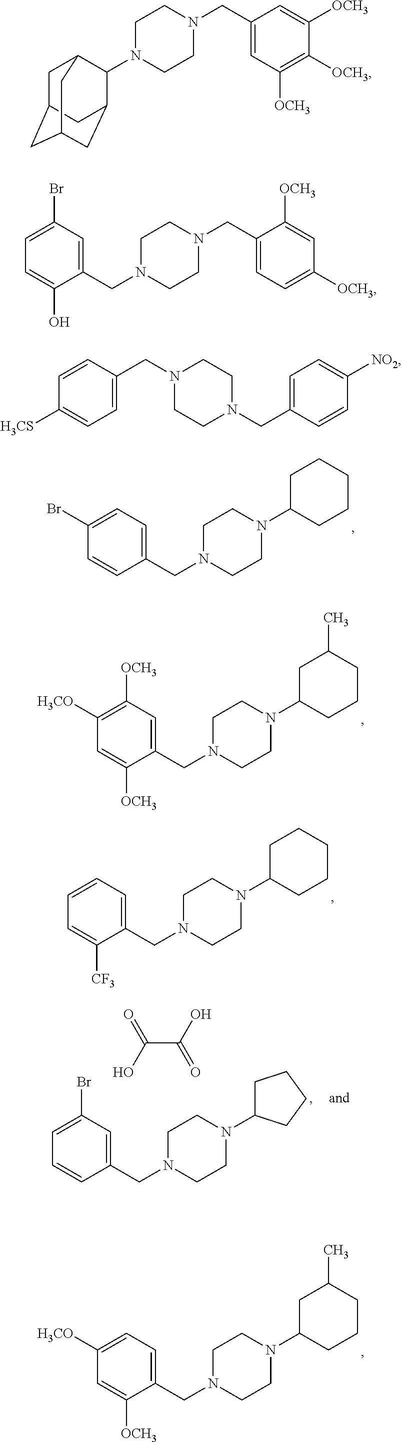
and pharmaceutically acceptable salts thereof.

15. A method of treating a neurodegenerative disease in a subject in need thereof, comprising administering to the subject a therapeutically effective amount of a compound of formula (II): 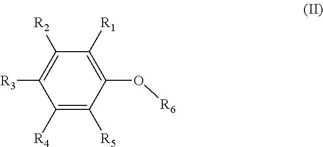
or a pharmaceutically acceptable salt thereof, wherein R₁, R₂, R₃, R₄, and R₅ are each independently selected from the group consisting of hydrogen, an alkyl, an alkenyl, an alkynyl, an aryl, an alkaryl, an aralkyl, a halo, a haloalkyl, hydroxy, an alkoxy, an alkenyloxy, an alkynyloxy, an aryloxy, acyloxy, a thioalkyl, an alkoxycarbonyl, an aryloxycarbonyl, a halocarbonyl, an alkylcarbonato, an arylcarbonato, a carboxy, a carboxylato, a carbamoyl, an amino, an alkylamido, an arylamido, an imino, an alkylimino, an arylimino, a nitro, and a nitroso; wherein R₁ and R₂, R₂ and R₃, R₃ and R₄, or R₄ and R₅ are optionally taken together with the carbon atoms to which they are attached to form a ring; and R⁶ is a group of formula —(CH₂)ₙ—(C(O))ₘ—NR^{a}R^{b}, wherein: m is 0 or 1; n is 1, 2, 3, 4, 5, or 6; and R^{a} and R^{b} are each independently selected from alkyl, or R^{a} and R^{b} are taken together with the nitrogen atom to which they are attached to form an optionally substituted heterocyclyl.

16. The method of claim 15, wherein the compound of formula (II) is a compound selected from the group consisting of: 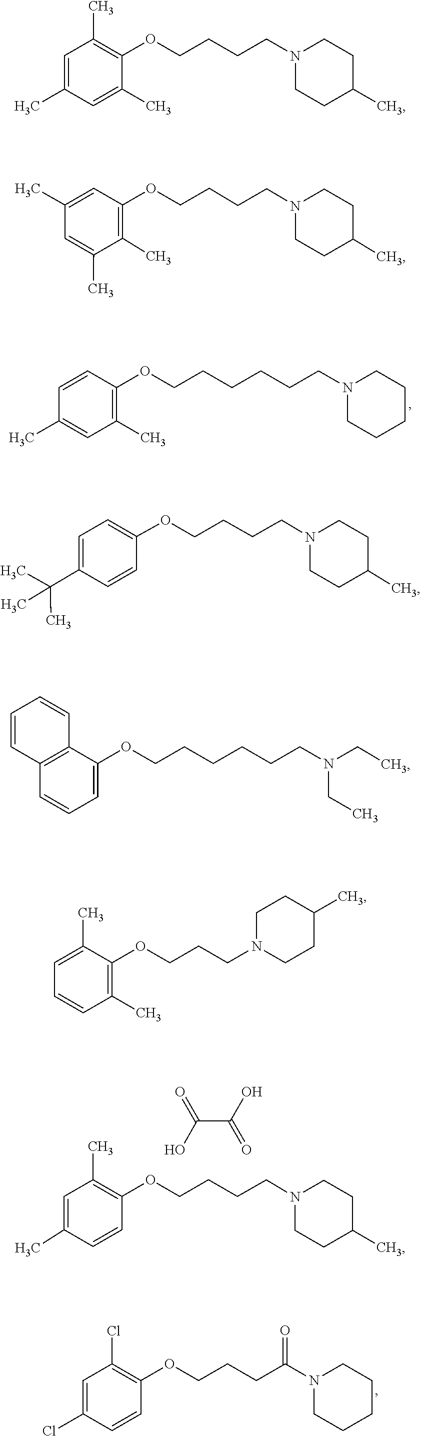 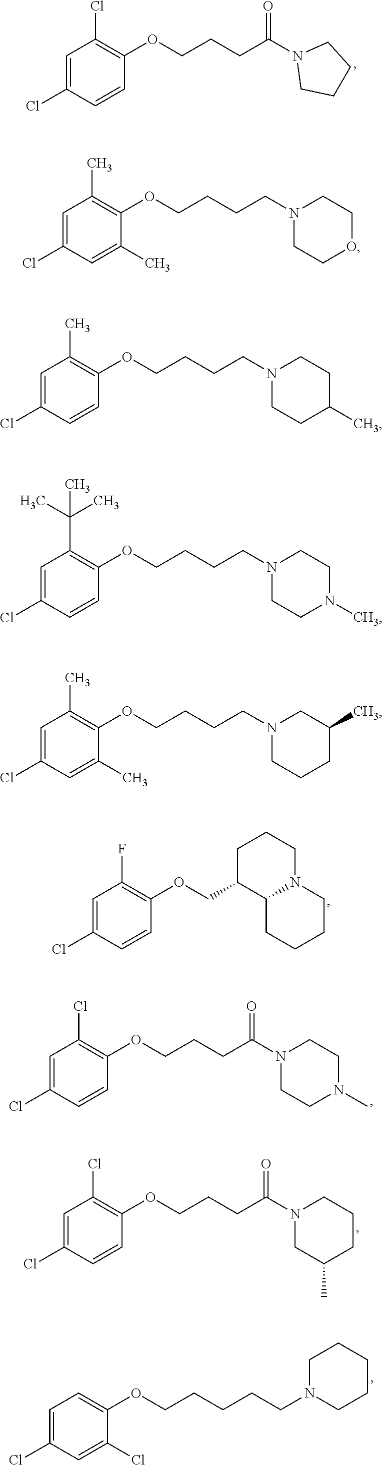 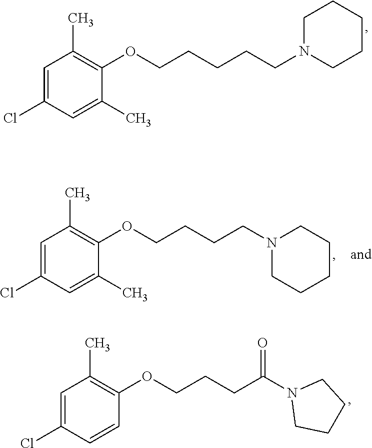 [SMILES: Cc1cc(Cl)cc(C)c1OCCCCCN1CCCCC1 ; Cc1cc(Cl)cc(C)c1OCCCCN1CCCCC1 ; Cc1cc(Cl)ccc1OCCCC(=O)N1CCCC1]
and pharmaceutically acceptable salts thereof.

17. A method of treating a neurodegenerative disease in a subject in need thereof, comprising administering to the subject a therapeutically effective amount of a compound of formula (III): 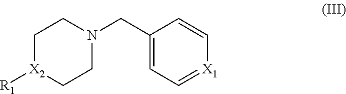
or a pharmaceutically acceptable salt thereof, wherein X₁ and X₂ are each independently selected from CH and N, and R₁ is selected from the group consisting of cycloalkyl and heterocyclyl, each of which is optionally substituted with 1 or 2 substituents.

18. The method of claim 17, wherein the compound of formula (III) is a compound selected from the group consisting of: 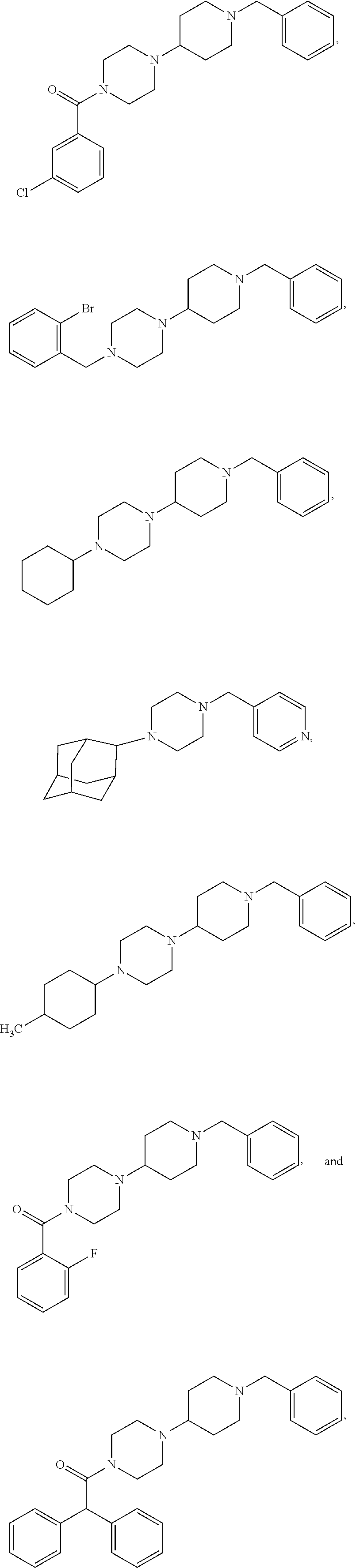
and pharmaceutically acceptable salts thereof.

19. The method of any one of claims 10-18, wherein the subject is a human.

20. The method of any one of claims 10-19, wherein the neurodegenerative disease is a demyelinating disease.

21. The method of claim 20, wherein the demyelinating disease is multiple sclerosis.

22. The method of any one of claims 10-21, further comprising administering an additional anti-neurodegenerative disease agent to the subject.

23. The method of claim 22, wherein the additional anti-neurodegenerative disease agent is selected from L-dopa, a cholinesterase inhibitor, an anticholinergic, a dopamine agonist, a steroid, and an immunomodulator.
